(19) United States
(12) Patent Application Publication
(10) Pub. No.: US 2025/0388859 A1
(43) Pub. Date: Dec. 25, 2025

(54) MULTI-WELL SYSTEMS AND METHODS FOR SORTING SPERM

(71) Applicant: COOPERSURGICAL, INC., Trumbull, CT (US)

(72) Inventors: William Thomas Sharp, Gaithersburg, MD (US); Utkan Demirci, Gaithersburg, MD (US)

(21) Appl. No.: 18/880,080

(22) PCT Filed: Jun. 30, 2023
(86) PCT No.: PCT/US2023/069553

§ 371 Date: Dec. 30, 2024

Related U.S. Application Data
(60) Provisional application No. 63/367,571, filed on Jul. 1, 2022.
(60) Provisional application No. 63/369,727, filed on Jul. 28, 2022.

(51) Int. Cl. C12N 5/071; C12M 1/00; C12M 1/26; C12M 3/00; C12M 3/06; C12N 5/076

(52) U.S. Cl. CPC C12N 5/0612; C12M 21/06; C12M 23/16; C12M 33/14; C12M 47/04; C12N 5/061

(57) ABSTRACT

Provided herein are systems and methods for sorting sperm that in illustrative embodiments include two or more separation channels each connected to a collection chamber. In some embodiments, at least one of the separation channels is attached to a filter chamber, which is also connected to an introduction channel. Some embodiments include a plurality of separation channels and connected collection chambers. Furthermore, in some embodiments the separation channels and connected collection chambers are arranged in parallel, in serial, or a combination thereof. Some embodiments herein include a pre-filter zone that can be a straight channel, or can be other than a straight channel, including a number of different disclosed configurations.

### CROSS-REFERENCE TO RELATED APPLICATIONS

This patent application claims priority to U.S. Provisional Application Ser. No. 63/367,571, filed Jul. 1, 2022, and Provisional Application Ser. No. 63/369,727, filed on Jul. 28, 2022. All of the applications cited in this paragraph are incorporated by reference herein in their entireties.

### TECHNICAL FIELD

The present disclosure relates generally to systems, devices and methods of sorting sperm.

### BACKGROUND OF THE INVENTION

According to estimates, there are more than 70 million infertile couples worldwide, and this number is increasing. Approximately 1 in every 4 infertile couples seek clinical treatment, where, according to sources, male factor (e.g., abnormal sperm production, motility, blockage of delivery of sperm or low sperm production) may account for about 50 percent of the infertility cases. To increase the probability of a successful conception and pregnancy, a variety of assisted reproductive technology procedures (ARTs), such as in vitro fertilization (IVF), intracytoplasmic sperm injection (ICSI), and artificial insemination (AI) such as intrauterine insemination (IUI), are generally utilized in reproductive clinics to treat infertile couples. ART can be life changing as such procedures provide the opportunity for some mothers and couples to have a child who otherwise might not have such opportunity.

To be performed successfully, each ART procedure requires a minimum number of healthy sperm, which are generally more motile and morphologically normal. For example, IUI usually requires at least 100,000 and more typically at least a million motile sperm, whereas IVF usually requires less than 1 million or even less than 100,000 motile sperm. ICSI requires just one healthy sperm, and isolating a single sperm for ICSI can be challenging with concentrated samples having a large number of sperm. Isolating (e.g., separating, concentrating or enriching) more motile and healthier sperm from the rest of a sperm sample, prior to using the sperm to perform an ART procedure, increases the chance of success of the ART procedure.

However, sorting does not guarantee ART success and sperm sample quality is variable not only between individual donors but even for the same donor at different time points. For example, some sperm samples may provide a sufficient number of healthy sperm for IUI, while other sperm samples from the same or a different donor may only provide enough healthy sperm for an ICSI procedure. Furthermore, it can be difficult to isolate just a single or a small number of sperm from a sample with millions of sperm, especially if they are in a relatively small volume. Furthermore, there are likely advantages to isolating the healthiest sperm from a sample. It is further noteworthy that ART procedures often need to be repeated to be successful, and for some females, IUI is attempted once or repeatedly and is not effective and thus IVF and/or ICSI are performed to try to yield a successful pregnancy. Thus, there is a need for a system and method that can be used to isolate multiple sorted sperm samples from the same initial sample, each of which can be used for a separate ART procedure of the same or a different type. Furthermore, there is a need for devices that have a further reduced risk when extracting sperm from the device, of producing a force that draws non-motile or poorly motile sperm up through the filter into a collection chamber. Finally, there is a need to isolate better swimming and healthier sperm even from samples that include motile sperm that swim through a separation filter and arrive at or near an outlet for extraction.

It would therefore be desirable to provide systems and methods for sorting or otherwise processing sperm that is robust and flexible, and therefore can be used to isolate or otherwise process relatively large numbers of healthy sperm when provided in a sperm sample, for certain ART procedures such as TUI, and/or to isolate one or a relatively small number of healthy or even the healthiest sperm for other ART procedures, such as ICSI. Furthermore, it would be helpful to provide systems and methods that permit such sorting and isolation/harvesting without prior knowledge of the quality of a sperm sample, and in illustrative embodiments without a predetermination of the ART procedure to be used with harvested sperm. It would further be desirable to provide systems and methods that can simultaneously isolate at least two separate sorted motile sperm samples, from one unsorted sperm sample, 1, 2 or more of which can optionally be frozen, and facilitates repeated ART procedures using motile sperm from the sample.

### SUMMARY OF THE INVENTION

The present invention overcomes the aforementioned drawbacks by providing systems and methods that are robust and flexible, and therefore can be used to isolate or otherwise effectively process sperm samples regardless of whether the sample has millions or 10s of millions of healthy, motile sperm or just a few healthy, motile sperm. The various channel and/or collection chamber configurations in systems disclosed herein allow for sorting and isolation of healthier and more motile sperm from all motile sperm in the sample. Thus, such method can be used as a sperm sorting system or device for sperm samples having as many as millions or as few as 1 sperm, and for virtually any downstream ART procedure.

In one aspect, provided herein is a system for sorting sperm, comprising:
a) a filter chamber comprising a filter comprising a plurality of micropores and arranged between a lower portion and an upper portion positioned above the lower portion; and
b) a post filter zone comprising a first post-filter separation channel connected to a first collection chamber configured to facilitate harvesting some of the motile sperm therein, wherein the post filter zone is other than a single straight separation channel connected to a single collection chamber. In some embodiments, the system further comprises an inlet connected either directly or indirectly to the filter chamber. In illustrative embodiments, the post filter zone has a plurality of separation channels each connected to a different one of a plurality of collection chambers. In some embodiments the filter is arranged horizontally within the filter chamber.

In one aspect, provided herein is a system for sorting sperm, comprising:
a) optionally a housing including a lower component and an upper component coupled together;
b) a fluidic system optionally supported by the housing;
c) an inlet, providing access to the fluidic system to deliver a sample comprising sperm to the fluidic system, and optionally extending through the lower component;
d) a filter chamber configured to pass motile sperm for harvesting and restrict non-motile sperm, the filter chamber comprising a filter comprising a plurality of micropores and arranged between a lower portion and an upper portion positioned above the lower portion;
e) optionally a pre-filter zone comprising at least one introduction channel extending from the inlet to the filter chamber to allow sperm delivered to the fluidic system through the inlet to progress along a fluidic path toward the filter chamber; and
f) a post filter zone comprising a first post-filter separation channel connected to a first collection chamber configured to facilitate harvesting some of the motile sperm therein, wherein either or both
   i) the pre-filter zone is other than a single straight channel; and
   ii) the post filter zone is other than a single straight separation channel connected to a single collection chamber. In some embodiments, the system comprises one or any combination of the optional elements recited in the aspect included in this paragraph, including in illustrative embodiments, all the optional elements. In illustrative embodiments, the post filter zone has a plurality of separation channels each connected to a different one of a plurality of collection chambers.

In one aspect, a system for sorting sperm is provided. The system includes a housing a lower component and an upper component coupled together; a fluidic system supported by the housing; an inlet that extends through the lower component and provides access to the fluidic system to deliver a sample comprising sperm to the fluidic system; a filter chamber configured to pass motile sperm for harvesting and restrict non-motile sperm, the filter chamber including a lower portion extending through the lower component and an upper portion extending through the upper component and positioned above the lower portion; a pre-filter zone comprising at least one introduction channel extending from the inlet to the filter chamber to allow sperm delivered to the fluidic system through the inlet to progress along a fluidic path toward the filter chamber; a filter including a plurality of micropores and arranged in the filter chamber to cause sperm traveling along the fluidic path to move through the filter, and in illustrative embodiments against gravity, to reach the upper portion; and a post filter zone comprising a first post-filter separation channel connected to a first collection chamber configured to facilitate harvesting some of the motile sperm therein, wherein either or both (i) the pre-filter zone is configured and/or adapted to sort sperm based on their swimming ability and is other than a single straight channel; and (ii) the post filter zone is configured and/or adapted to sort sperm based on their swimming ability and is other than a single straight separation channel connected to a single collection chamber.

In another aspect, a system for sorting sperm is provided, including a housing including a lower component and an upper component coupled together; a fluidic system supported by the housing; an inlet extending through the lower component and providing access to the fluidic system to deliver a sample comprising sperm to the fluidic system; a filter chamber configured to pass motile sperm for harvesting and restrict non-motile sperm, the filter chamber including a lower portion extending through the lower component and an upper portion extending through the upper component and positioned above the lower portion; at least one introduction channel extending from the inlet to the filter chamber to allow sperm delivered to the fluidic system through the inlet to progress along a fluidic path toward the filter chamber; a filter including a plurality of micropores and arranged in the filter chamber to cause sperm traveling along the fluidic path to move through the filter, and in illustrative embodiments against gravity, to reach the upper portion; and a post-filter zone comprising: (i) a first separation channel connecting the upper portion to a first collection chamber, the first collection chamber being configured to facilitate harvesting some of the motile sperm therein; and (ii) a second separation channel connected to a second collection chamber, the second collection chamber being configured to facilitate harvesting some of the motile sperm therein, wherein the second separation channel is connected to either the upper portion or the first collection chamber, wherein the first separation channel has a length of less than 5 mm, wherein the second separation channel has a length between 5 and 20 mm, and wherein the length of the second separation channel is at least 1.5 times the length of the first separation channel.

Illustrative systems and methods include an inlet into which a crude, unprocessed sperm sample can be loaded. Motile sperm in the sample travel through an introduction channel to a filter chamber in which some of the motile sperm swim through a filter, and then into two or more collection chambers that are fluidly connected directly or indirectly to the filter chamber. Motile sperm are typically harvested from one or more of the collection chambers.

Accordingly, in yet another aspect, a method for sorting sperm is provided, including the steps of delivering a sample comprising sperm into an inlet connected to a fluidic system; allowing sperm in the sample of sperm to traverse a fluidic path through the fluidic system from the inlet through at least one introduction channel into a filter chamber, wherein the filter chamber includes a lower portion and an upper portion positioned above the lower portion, and wherein the lower portion and the upper portion are separated by a filter positioned in the filter chamber, wherein the filter has micropores sized to permit a head of the sperm to pass therethrough, in illustrative embodiments, without the application of an external force, allowing at least some motile sperm from the sample of sperm that have entered the lower portion of the filter chamber to selectively pass through the filter, and in illustrative embodiments against gravity, so as to traverse the fluidic path into the upper portion of the filter chamber, and then to traverse the fluidic path through a first separation channel into a first collection chamber such that a percentage of motile sperm present within the first collection chamber is greater than a percentage of motile sperm within the sample; either i) allowing at least one of the motile sperm in the first collection chamber to further traverse through a second separation channel into a second collection chamber, in illustrative embodiments without the application of an external force; or ii) allowing at least one of the motile sperm from the upper portion to traverse through a second separation channel connected directly to the upper portion of the filter chamber, into a second collection chamber, in illustrative embodiments without the application of an external force; and harvesting at least some of the motile sperm that have passed into one or both the first collection chamber and the second collection chamber. In illustrative embodiments the second separation channel is at least 1.5, 2, 3, 4, 5, or 10× the length of the first separation channel.

In yet another aspect, a method for sorting sperm is provided, including delivering a sample comprising sperm to an inlet connected to a fluidic system comprising the inlet, a pre-filter zone comprising at least one introduction channel extending from the inlet to a filter chamber, a filter having a plurality of micropores and arranged in the filter chamber; and a post filter zone comprising a first post-filter separation channel connected to a first collection chamber configured to facilitate harvesting some of the motile sperm therein, wherein either or both (i) the pre-filter zone is configured and/or adapted to sort sperm based on their swimming ability and is other than a single straight channel; and (ii) the post filter zone is configured and/or adapted to sort sperm based on their swimming ability and is other than a single straight separation channel connected to a single collection chamber; allowing sperm delivered to the fluidic system through the inlet to progress along a fluidic path toward the filter chamber, in illustrative embodiments without the application of an external force, and then to move through the filter and against gravity to the post-filter zone, and then to move through the post-filter zone to the first collection chamber; and harvesting at least some motile sperm from a collection chamber in the post-filter zone.

Further details regarding aspects and embodiments of the present disclosure are provided throughout this patent application. Sections and section headers are for ease of reading and are not intended to limit combinations of disclosure, such as methods, compositions, and kits or functional elements therein across sections. Further details regarding aspects and embodiments of the present disclosure are provided throughout this patent application. Sections and section headers are for ease of reading and are not intended to limit combinations of disclosure, such as methods, compositions, or other functional elements therein across sections.

### BRIEF DESCRIPTION OF THE DRAWINGS

FIG. 1A is a cross-sectional view of a multi-well system 110 for sorting sperm 01 in an illustrative embodiment, with first and second separation channels 127a, 127b and first and second collection chambers 125a, 125b arranged in serial.

FIG. 1B is a top view of the system 110 of FIG. 1A.

FIG. 2 is a top view of a multi-well system 210 for sorting sperm 01 in another illustrative embodiment, with the first separation channel 227a and the first collection chamber 225a separate from the second separation channel 227b and the second collection chamber 225b.

FIG. 3 is a top view of a multi-well system 310 for sorting sperm 01 in another illustrative embodiment, with a plurality of parallel separation channels (i.e., 327b, 327c, 327d, 327e, 327f) fluidly joining the first collection chamber 325a with the second collection chamber 325b. A portion of the illustration is enlarged for easier viewing.

FIG. 4 is atop view of a multi-well system 410 for sorting sperm 01 in another illustrative embodiment, with the introduction channel 420 including a curve.

FIG. 5 is atop view of a multi-well system 510 for sorting sperm 01 in another illustrative embodiment, wherein the post-filter zone 528 includes more than two separation channels and more than two collection chambers in an alternating, serial arrangement.

FIG. 6 is a top view of a multi-well system 610 for sorting sperm 01 in another illustrative embodiment, wherein the post-filter zone 628 includes more than four separation channels and more than four collection chambers arranged as the spokes of a wheel.

FIG. 7 is a top view of a multi-well system 710 for sorting sperm 01 in another illustrative embodiment, with a curved separation channel 727b.

As used herein, the term “about” refers to a value 10% less or 10% more than the disclosed value.

For example, “about 1% sucrose” would include 0.9% to 1.1% sucrose.

It is to be understood that the present disclosure and the aspects and embodiments provided herein, are not limited to particular examples disclosed, as such may, of course, vary. It is also to be understood that the terminology used herein is for the purpose of disclosing particular examples and embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within embodiments herein, subject to any specifically excluded limit in the stated range. Where the stated range includes one or both of the limits, ranges excluding either or both of those included limits are also included in the embodiments herein. When multiple low and multiple high values for ranges are given that overlap, a skilled artisan will recognize that a selected range will include a low value that is less than the high value. All headings in this specification are for the convenience of the reader and are not limiting.

Unless defined otherwise, all technical and scientific terms used herein have the same meaning as commonly understood by one of ordinary skill in the art to which the aspects and embodiments provided herein belong. Although any methods and materials similar or equivalent to those described herein can also be used in the practice or testing of any aspects or embodiments provided herein, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a chimeric antigen receptor” includes a plurality of such chimeric antigen receptors and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless specifically stated or otherwise obvious from context, as used herein, the term “or” is understood to be inclusive. The term “and/or” as used in a phrase such as “A and/or B” herein includes each of the following: A and B; A or B; A (alone); and B (alone). Similarly, the term “and/or” as used in a phrase such as “A, B, and/or C” includes each of the following: A, B, and C; A, B, or C; A or B; A or C; B or C; A and B; A and C; B and C; A (alone); B (alone); and C (alone). This logic extends to any number of items in a list that are connected with the term “and/or”.

It is appreciated that certain features of aspects and embodiments herein, which are, for clarity, discussed in the context of separate embodiments, may also be provided in combination in a single embodiment. Conversely, various aspects and embodiments, which are, for brevity, discussed in the context of a single aspect or embodiment, may also be provided separately or in any suitable sub-combination. All combinations of aspects and embodiments are specifically embraced herein and are disclosed herein just as if each and every combination was individually and explicitly disclosed. In addition, all sub-combinations of the various aspects and embodiments and elements thereof are also specifically disclosed herein even if each and every such sub-combination is not individually and explicitly disclosed herein. Furthermore, element numbers corresponding to figure elements are provided in the discussion of certain aspects and embodiments herein. It will be understood that the various embodiments illustrated in the figures have corresponding elements between them, sometimes with numbers that correspond to similar structures in different embodiments. In some cases such numbers of corresponding elements in different figures, share the same last 2 digits. Furthermore, it will be understood that reference to a certain element number in this disclosure is for non-limiting exemplary purposes only, and should not be taken as limiting to only the corresponding illustrated embodiment in the figure that contains such element.

### DETAILED DESCRIPTION

Provided herein in certain aspects are macrofluidic, mesofluidic, microfluidic, macro-micro or meso-micro fluidic sperm sorting (MMSS) systems, devices and related methods that are effective for, adapted to, and/or configured to sort sperm efficiently, reliably, and successfully and in illustrative embodiments, that are effective for, adapted for, and/or configured to isolate or otherwise process sperm samples, in illustrative embodiments semen samples comprising sperm, regardless of whether a sample has millions or 10s of millions of healthy sperm or just a few healthy sperm. Thus, such systems and methods can be effectively used to harvest sperm for a wide variety of downstream ART procedures. Accordingly, using such system and methods, healthy motile sperm typically are harvested/collected at the outlets, or collection chamber(s), post-sorting and in some embodiments used in downstream ART procedures. Systems and methods herein increase the robustness of sperm separation such that sperm samples that vary greatly in the percent and number of healthy, motile sperm can more frequently be successfully used for ART procedures after sorting sperm using these systems and methods herein. Furthermore, the systems and methods herein can be used to isolate multiple sorted samples from the same initial sample in the same sorting run or sorting method performance. Depending on the specific embodiment herein employed, multiple sorted samples can be sorted and optionally detected and/or in illustrative embodiments harvested with similar (e.g. +/−20, 15, 10, or 5%) numbers of motile sperm (i.e., motile sperm counts) between them, or very different (e.g. 1 or more samples having 25%, 50% or 100%, or 1.5, 2, 3, 4, 5, 6, 7, 8, 9, 10, 100, 500, 1,000, 5,000, 10,000, 100,000, 1,000,000, 2,000,000, 3,000,000,000, 4,000,000,000, or 5,000,000 times more) motile sperm counts between them. In illustrative embodiments, multiple chambers and optionally channels therebetween allow for collection of motile sperm that have swam to the collection chamber in a flow-free manner (i.e., without an external force). The further the chamber(s) from which sperm are collected is from the filter chamber, the less vacuum or force is produced by syringe or pipet removal of the sperm, thereby reducing the risk of pulling less-motile as well as dead and other non-motile sperm from below the filter.

Accordingly, provided herein in certain illustrative aspects are systems and methods for sorting sperm that include two or more separation channels each connected to a collection chamber wherein at least one of the separation channels is attached to a filter chamber, which is optionally connected to an introduction channel. Some embodiments include a plurality of separation channels and connected collection chambers. Furthermore, in some embodiments the separation channels and connected collection chambers are arranged in parallel, in serial, or a combination thereof.

Accordingly, in one aspect, provided herein is a system for sorting sperm, comprising:
a) a filter chamber comprising a filter comprising a plurality of micropores and arranged between a lower portion and an upper portion positioned above the lower portion; and
b) a post filter zone comprising a first post-filter separation channel connected to a first collection chamber configured to facilitate harvesting some of the motile sperm therein, wherein the post filter zone is other than a single straight separation channel connected to a single collection chamber. In some embodiments, the system further comprises an inlet connected either directly or indirectly to the filter chamber. In illustrative embodiments, the post filter zone has a plurality of separation channels each connected to a different one of a plurality of collection chambers.

In one aspect, provided herein is a system for sorting sperm, comprising:
a) optionally a housing including a lower component and an upper component coupled together;
b) a fluidic system optionally supported by the housing;
c) an inlet, providing access to the fluidic system to deliver a sample comprising sperm to the fluidic system, and optionally extending through the lower component;
d) a filter chamber configured to pass motile sperm for harvesting and restrict non-motile sperm, the filter chamber comprising a filter comprising a plurality of micropores and arranged between a lower portion and an upper portion positioned above the lower portion;
e) optionally a pre-filter zone comprising at least one introduction channel extending from the inlet to the filter chamber to allow sperm delivered to the fluidic system through the inlet to progress along a fluidic path toward the filter chamber; and
f) a post filter zone comprising a first post-filter separation channel connected to a first collection chamber configured to facilitate harvesting some of the motile sperm therein, wherein either or both
   i) the pre-filter zone is other than a single straight channel; and
   ii) the post filter zone is other than a single straight separation channel connected to a single collection chamber. In some embodiments, the system comprises one or any combination of the optional elements recited in the aspect included in this paragraph, including in illustrative embodiment, all the optional elements. In illustrative embodiments, the post filter zone has a plurality of separation channels each connected to a different one of a plurality of collection chambers.

In one aspect, provided herein is a system or device for sorting sperm that includes a housing having a lower component and an upper component, which in some embodiments, can be a lower component layer and upper component layer, respectively, coupled together; a fluidic system supported by the housing; an optional inlet that extends through the lower component and provides access to the fluidic system to deliver a sample comprising sperm to the fluidic system; a filter chamber configured to pass, adapted to pass and/or effective for passing motile sperm for harvesting and in illustrative embodiments restrict non-motile sperm. The filter chamber includes a lower portion, which in certain illustrative embodiments extends through the lower component, when present, and an upper portion, which in certain illustrative embodiments extends through the upper component, when present, and is positioned above the lower portion. The system or device typically further includes an optional pre-filter zone that includes at least one introduction channel extending from the inlet, when present, to the filter chamber to allow sperm delivered to the fluidic system or device through the inlet to progress along a fluidic path toward the filter chamber; a filter including a plurality of micropores and arranged in the filter chamber to cause sperm traveling along the fluidic path to move through the filter and typically against gravity to reach the upper portion; and a post filter zone comprising a first post-filter separation channel connected to a first collection chamber configured to facilitate harvesting at least some of the motile sperm therein. In illustrative embodiments either or both (i) the pre-filter zone is configured and/or adapted to sort sperm based on their swimming ability and is other than a single straight channel; and (ii) the post filter zone is configured and/or adapted to sort sperm based on their swimming ability and is other than a single straight separation channel connected to a single collection chamber. In some embodiments of the aspect provided in this paragraph, the system or device does not include one of, or both the inlet and the pre-filter zone. In illustrative embodiments of this aspect, the system or device includes both the inlet and the pre-filter zone.

In another aspect, a system or device for sorting sperm is provided, including in illustrative embodiments, a housing including a lower component and an upper component, which in some embodiments, can be a lower component layer and upper component layer, respectively, coupled together; a fluidic system supported by the housing; an optional inlet extending through the lower component and providing access to the fluidic system to deliver a sample comprising sperm to the fluidic system. The system or device further typically includes a filter chamber configured to pass motile sperm for harvesting and restrict non-motile sperm, the filter chamber including a lower portion, which in certain illustrative embodiments extends through the lower component, when present, and an upper portion, which in certain illustrative embodiments extends through the upper component, when present, and is positioned above the lower portion; at least one optional introduction channel extending from the inlet, when present, to the filter chamber to allow sperm delivered to the fluidic system through the inlet to progress along a fluidic path toward the filter chamber; a filter including a plurality of micropores and arranged in the filter chamber to cause sperm traveling along the fluidic path to move through the filter and against gravity to reach the upper portion; and a post-filter zone that in certain illustrative aspects includes: (i) a first separation channel connecting the upper portion to a first collection chamber, the first collection chamber being configured to facilitate harvesting some of the motile sperm therein; and (ii) a second separation channel connected to a second collection chamber, the second collection chamber being configured to facilitate harvesting at least some of the motile sperm therein. In illustrative embodiments the second separation channel is connected to either the upper portion or the first collection chamber, wherein the first separation channel has a length that is less than, for example ¾ ½, ¼, ⅔ or ⅓, the length of the second separation channel. As a non-limiting example the first separation channel can have a length of less than 5 mm, wherein the second separation channel has a length between 5 and 20 mm, and in illustrative embodiments wherein the length of the second separation channel is at least 1.1, 1.25, 1.5, 1.75, 2, 3, 4, or 5 times the length of the first separation channel. In some embodiments of the aspect provided in this paragraph, the system or device does not include one of, or both the inlet and the pre-filter zone. In illustrative embodiments of this aspect, the system or device includes both the inlet and the pre-filter zone.

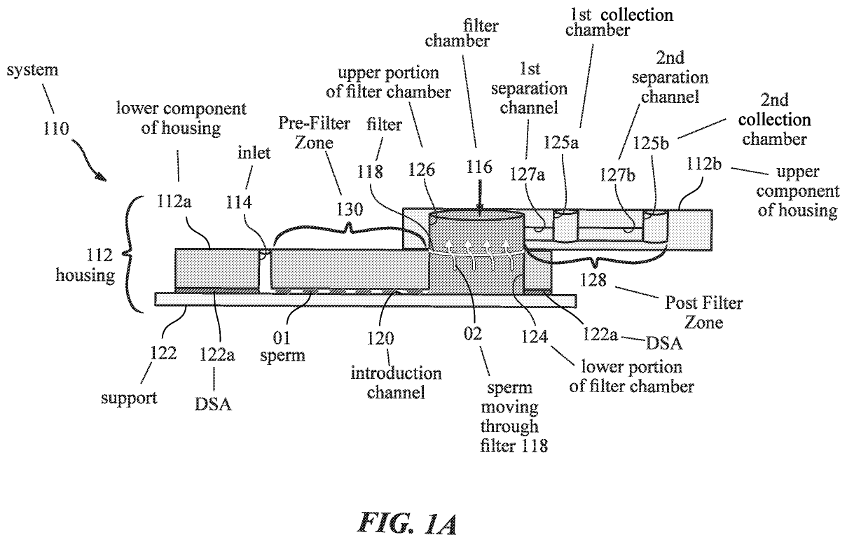

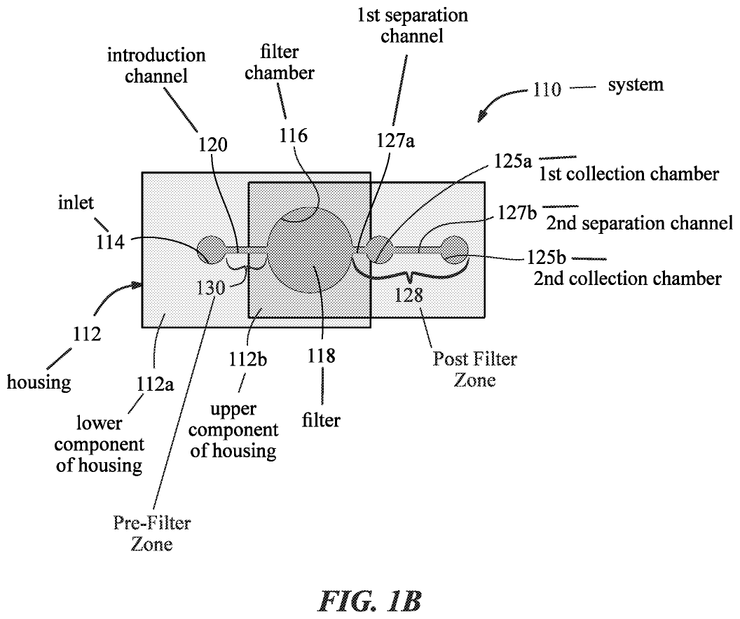

In an illustrative embodiment, the system is configured and/or arranged such that the filter is at an angle relative to the longitudinal axis of the system 110 and/or the support 122. For example, in FIG. 1A, the filter 118 is substantially parallel with the longitudinal axis of the system 110 and the support 122, and substantially perpendicular to the central axis (e.g., longitudinal axis) of the filter chamber 116. In certain illustrative embodiments, the filter chamber 116 is arranged and/or configured such that its central axis is oriented at an angle relative to the plane of the support 122, wherein the angle is greater than or less than 90-degrees. In some illustrated embodiments, such angle can range from between ±10-degrees (e.g., relative to the longitudinal axis of the support 122) at the low end of the range, to ±20, ±30, ±45, ±50, ±60, ±75, ±80 or ±85 degrees at the high end. In some illustrated embodiments, the sperm are sorted by the filter 118, and do not swim against gravity.

### Multi-Well System for Sorting Sperm

FIGS. 1(A and B) to 7 illustrate exemplary embodiments of multi-well systems configured and/or adapted for sorting sperm, especially sorting sperm based upon their swimming ability, such that the most motile, healthiest sperm can be harvested for use in various ART procedures. These procedures include but are not limited to in vivo artificial insemination (AI) procedures, such as for example intrauterine insemination (IUI), in vitro fertilization (IVF), and intracytoplasmic sperm injection (ICSI). Systems provided herein typically include a pre-filter zone and a post-filter zone, which are discussed below, separated by a filter chamber. Illustrative embodiments of such systems include a post-filter zone that includes two or more (e.g. a plurality of) separation channels and collection chambers connected to the filter chamber.

### System Housing and Filter Chamber

Systems and devices disclosed herein typically include a housing that is typically a rigid structure onto which the various channels, chambers, and outlets disclosed herein are formed. Typically, the housing includes an upper component and a lower component. In some embodiments the housing includes a substrate below the lower component, and in illustrative embodiments having an upper surface that forms or is in contact with the lower surface of the lower component through most, substantially all, or all of the length of the lower surface of the lower component.

The lower component in certain illustrative embodiments, is a lower layer, also referred to as a lower component layer, and the upper component is an upper layer, also referred to as an upper component layer. In such embodiments, the lower component layer and the upper component layer are coupled, or joined together.

The lower component, which in illustrative embodiments is a lower layer and the upper component, which in illustrative embodiments is an upper layer, when coupled together, form a collection chamber (i.e., filter chamber) having a bottom chamber (i.e., bottom portion or first chamber), and an upper chamber (i.e., top portion or second chamber), and comprise a filter arranged therebetween that separates the lower chamber and the upper chamber. In some embodiments, the bottom chamber (i.e., first chamber) extends through the lower component layer, and the upper chamber (i.e., second chamber) is positioned above the first chamber and extends through the upper component layer. In these embodiments, a substrate or support can form the bottom surface of the filter chamber. In some embodiments, the filter is located between the upper component (e.g., upper layer) and the lower component (e.g., upper layer).

A device or system herein optionally includes an inlet which can extend through the upper component, the lower component, or both. The inlet when present, provides access to the lower portion of the filter chamber typically via an introduction channel, which forms a channel between the inlet and the lower portion of the filter chamber.

FIGS. 1A-1B illustrate a sperm sorting system 110 in an exemplary embodiment provided herein. The system 110 includes a housing 112 having an inlet 114 and a filter chamber 116 having a filter 118 arranged therein. The filter 118 can be a polycarbonate filter or other filter having suitable materials properties, such as but not limited to polyester or nylon, such as pore or passage size, as will be discussed. The housing 112 includes a lower component 112a attached to a support 122, also referred to as a substrate. An upper component 112b is joined with the lower component 112a, such as by any means known in the art. For example, the housing 112 can be constructed of a polydimethylsiloxane-(PDMS), poly(methyl methacrylate) (PMMA, 3 mm thick; McMaster Carr, Atlanta, GA), as non-limiting examples. Other plastic materials that can be used include copolyester, polycarbonate, and ABS, for example. Adhesives, such as double side adhesive 122a (DSA, 120 μm thick, St. Paul, MN), which can be cut using a laser cutter (Versa Laser™, Scottsdale, AZ) such as to form the channel 120 and circular portions for the inlet 114 and the filter chamber 116, can be used to join the upper portion 112b and the lower portion 112a. For example, in an illustrative embodiment, the lower component 112a of the housing 112 can include one 3 mm PMMA section cut to an area of 50 mm×30 mm and the upper component 112b can include a second 3 mm PMMA section cut to an area of 30 mm×30 mm. In some embodiments, at least some of the channels or chambers of the system or device can be formed using a cavity mold process, such as a cold runner system.

In illustrative embodiments, the support 122 is rectangular in shape, for example, having the approximate size and shape of a microscope slide, and in some embodiments is a microchip, or a microfluidic chip. Thus, in some embodiments, the system is a microfluidic system, in some embodiments it is a macrofluidic system and in some embodiments some portions (e.g., chamber(s) and channel(s)) are microfluidic and have dimensions in micrometers and/or microliters, and some portions (e.g., chamber(s) and channel(s)) are microfluidic and have larger dimensions. Various non-limiting exemplary dimensions for a device or system herein, or chambers and channels therein, are provided hereinbelow.

The filter chamber can be open or closed at the top. In illustrative embodiments there is one or more ports through the top channel of the filter chamber. Such ports can be open to the ambient or can be connected to a subassembly for providing a positive or negative force. In some embodiments, the top of the filter chamber can be covered by a lid, which can be arranged such that it can be open or closed by an operator of the system or device. Alternatively, the top of the filter chamber can be closed with an adhesive seal that attaches a top portion or lid to the side walls of the filter chamber, which is not intended to be open or closed during intended use.

Referring to the non-limiting exemplary system illustrated in FIGS. 1A-1B, the lower component 112a is attached to the support 122, so as to provide an introduction channel 120 between the lower component 112a and the support 122, so as to fluidly connect the inlet 114 with the filter chamber 116, and thereby to allow for movement (i.e., swimming) of sperm in a sample of sperm 01 along a fluidic path from the inlet 114 and into the filter chamber 116. The inlet 114 forms an orifice located in an upper surface of the lower component 112a and extends toward the support 122 such that the inlet 114 is fluidly connected with the introduction channel 120. The filter chamber 116 extends upwardly from the support 122, through the lower component 112a and the upper component 112b, in illustrative embodiments to an additional orifice in an upper surface of the upper component 112b. In such non-limiting embodiments that include an orifice, the filter chamber 116 can be accessed therethrough, such as for withdrawal or harvesting of a portion of sperm 01 (e.g., sorted sperm) therein. In a non-limiting illustrative embodiment, a 0.6 mm inlet 114 is cut into the lower component 112a at a 5 mm distance from the lower portion 124 of the filter chamber (116). Cylinders for example of 20 mm diameter can be cut into the upper and lower components 112b and 112a, so as to form the lower and upper portions 124, 126, respectively, of filter chamber 116. The lower component 112a can be attached to a support 122, such as but not limited to a glass slide, such as by using DSA 122a. The upper portion 126 is aligned with the lower portion 124, and then the upper component 112b can be attached to the lower component 112a, such as by using DSA 122a. In some illustrative embodiment, the system 110 can be disposable and configured to, adapted to, and capable of holding a liquid semen sample comprising sperm (either fresh or frozen, processed or raw), for example of 10 μl-10 ml in volume. For example, the sample of sperm, in illustrative embodiments, can be a liquid semen sample of sperm (either fresh or frozen, processed or raw). For example, in some illustrative embodiments, the system 110 is configured to, effective for, adapted to, and capable of receiving and/or holding a liquid semen sample comprising sperm (either fresh or frozen, processed or raw) that has a volume ranging from about 50 μl at the low end of the range and 250 μl, 500 μl, 750 μl, 1 ml, 2 ml and 5 ml at the high end of the range. In some illustrative embodiments, the volume of the liquid semen sample is even larger, ranging from 5.5 ml at the low end of the range and 9.9 ml at the high end of the range. As noted supra, one or more of the inlet 114 and the pre-filter zone 130 (e.g. the introduction channel 120) are optional in certain embodiments. In one such embodiment, the inlet 114 and the pre-filter zone 130 are omitted. Accordingly, in some examples of such embodiment, no inlet is cut into the housing lower component 112a and only a circle (e.g., no shapes corresponding to an inlet or an introduction channel) is cut into the DSA so as to align with the cylinder cut into the lower component 112a (e.g., the cylinder forms the lower portion 124 of the filter chamber 116) when the support 122 is attached to the lower component 112a. In such embodiment, the lower component 112a can be configured and arranged with an opening therethrough so as to provide direct access to the filter chamber lower portion 124 (e.g., when the housing 112 cannot be opened) or the housing 112 can be configured and arranged to be openable such that a sperm sample (e.g., a sample of sperm to be sorted) can be added by an operator such as a medical professional or a consumer, directly to the filter chamber lower portion 124. In certain embodiments, the housing lower component 112a can include a reservoir, such as but not limited to below the filter chamber lower portion 124, for receipt of the sperm sample. In certain illustrative embodiments the inlet, introduction channel and pre-filter zone are present in a device or system herein.

Still referring to FIGS. 1A-1B, the introduction channel 120 extends from the inlet 114 to the filter chamber 116. The filter chamber 116 includes a lower portion 124 and an upper portion 126. The lower portion 124 is located proximate to the support 122 and the upper portion 126 is located distally with respect to the support 122, above the lower portion 124. As will be discussed, the lower portion 124 is designed to collect the semen of a sample, whether fresh or frozen, processed or raw, that has been presented to the inlet 114 and the upper portion 126 is designed to filter the motile sperms.

The filter 118, which includes a plurality of micropores, is arranged in the filter chamber 116, between the upper and lower components 112b, 112a, respectively. As sperm travelling along the flow path move from the lower portion 124 to the upper portion 126, the sperm must move through the filter 118 (e.g., such as through the micropores sized to allow the head of a sperm to pass therethrough, as denoted by the white arrows 02) and also upward against gravity. Thus, non-motile sperm are restricted by the filter 118 and gravity while motile sperm continue to move or swim along the flow path, which separates the sperm based upon their swimming ability and therefore their health. The system 110 can be a polydimethylsiloxane-(PDMS) based, polymethylmethacrylate-(PMMA) based, or other system.

### Pre-Filter Zone

In illustrative embodiments, the systems and devices herein include a pre-filter zone that that extends from the inlet to the lower portion of the filter chamber and includes the introduction channel. The inlet can be connected to the lower portion of the filter chamber by a single straight channel (i.e., directly) or by other than a single, straight channel (i.e., indirectly). In certain embodiments, the pre-filter zone is configured and/or adapted to sort or to help sort, the more motile sperm and/or morphologically normal sperm t from the less motile and/or morphologically abnormal sperm in a sample that is added into the device through the inlet port. The pre-filter zone can comprise a parallel arrangement of channels such that different sperm in a sample applied to the inlet can take different paths through different channels within the pre-filter zone, or the system or device can be configured such that the channel(s) of the pre-filter zone are arranged such that there is a single path through the filter zone.

Accordingly, in certain illustrative embodiments, such as for example but not limited to with reference to FIGS. 1A-1B, the introduction channel 120 can include structures adapted for enhancing the sorting of the more motile, morphologically normal sperm from the less motile and/or morphologically abnormal sperm and/or from cellular debris and/or other structures/materials. For example, in an illustrative embodiment, the introduction channel 120 can include dam-like structures (i.e., dams), partial walls or similar structures, that are adapted to retain sperm that are not able to swim over them, dead sperm and/or cellular debris. In another illustrative embodiment, the introduction channel 120 can include one or more, and typically a plurality or set of pillars of various shapes (e.g. cylindrical, square, rectangular), sizes (e.g. between 1/10 on the low end of the range and ⅕, ¼, ⅓, ½, ⅔, ¾, and ⅘ on the high end of the range, of the diameter, area, and/or width of a channel, or between ⅕ on the low end of the range and ¼, ⅓, ½, ⅔, ¾, and ⅘ on the high end of the range, of the diameter, area, and/or width of a channel) and spacing (e.g. between 100 μm on the low end of the range and 250 μm, 500 μm, 1 mm, 2 mm, and 5 mm on the high end of the range between the center of pillars in the channel, or between 250 μm on the low end of the range and 500 μm, 1 mm, 2 mm, and 5 mm on the high end of the range between the center of pillars in the channel), configured to, effective for, and adapted to modify the path of the sperm as they swim through the introduction channel 120 and thereby help to separate the more motile sperm and/or morphologically normal sperm from less motile and/or morphologically abnormal sperm as they swim toward the filter chamber 116. Furthermore, a system or device herein can have pillars in the lower portion of the filter chamber (i.e., lower chamber) below the filter to help support the filter for example in a horizontal arrangement within the device when the device is laid flat on a surface.

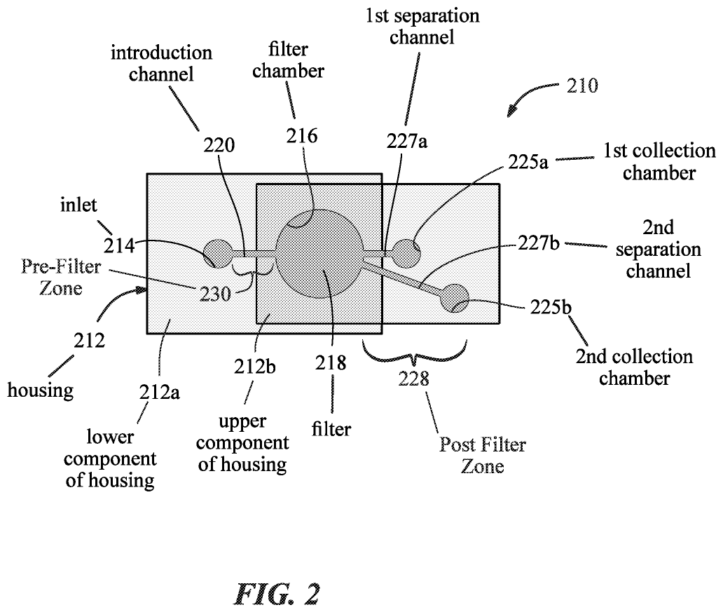

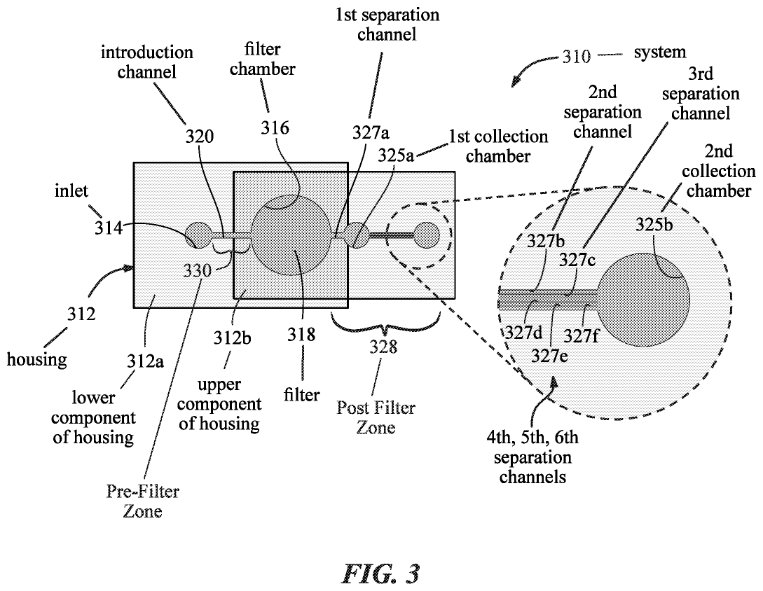

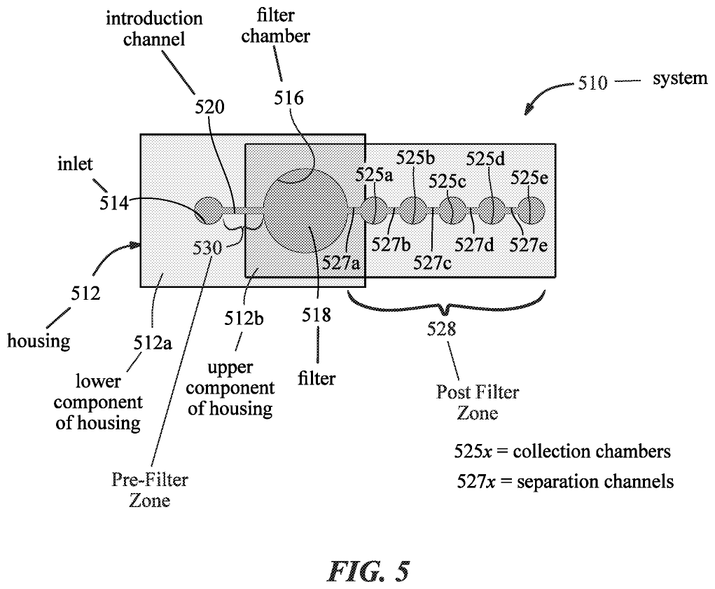

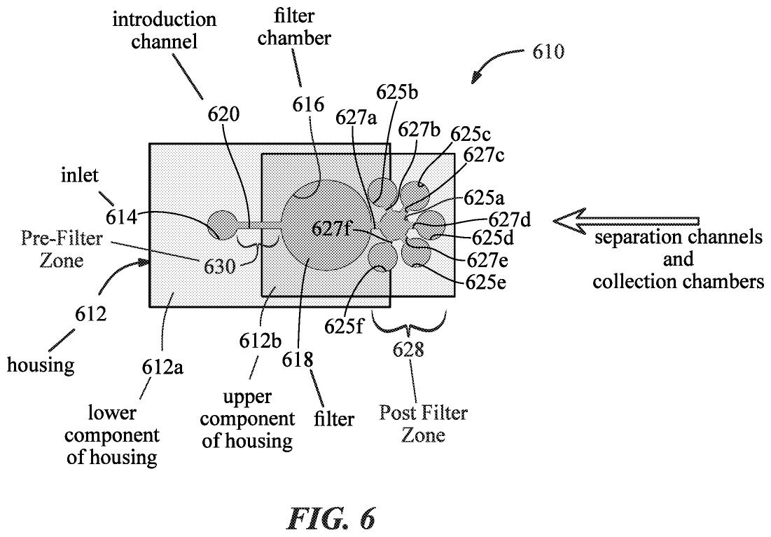

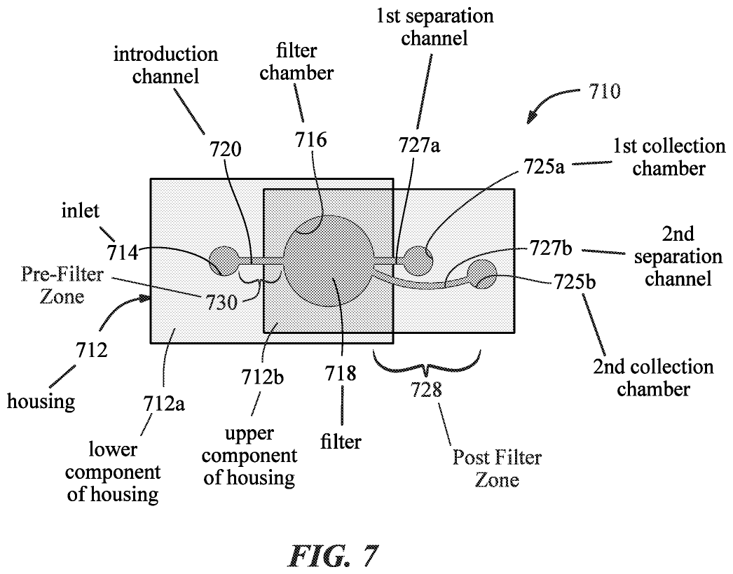

In certain illustrative embodiments, such as are shown in FIGS. 1A-3 and 5-7, the pre-filter zone comprises a straight channel, such as introduction channel, with structures that assist in sorting the sperm, such as dams, pillars and the like discussed above. For example, as shown in FIGS. 1A-1B, the system 110 includes a straight pre-filter zone 130 with an introduction channel 120 extending from the inlet 114 to the filter chamber 116, so as to allow sperm delivered to the fluidic system through the inlet 114 to progress or swim along a fluidic path toward the filter chamber 116. In another illustrative embodiment shown in FIG. 2, the system 210 includes a pre-filter zone 230 includes an introduction channel 220 extending from an inlet 214 to a filter chamber 216, so as to allow sperm delivered to the fluidic system through the inlet 214 to progress or swim along a fluidic path toward the filter chamber 216. In another illustrative embodiment shown in FIG. 3, the system 310 includes a pre-filter zone 330 includes an introduction channel 320 extending from an inlet 314 to a filter chamber 316, so as to allow sperm delivered to the fluidic system through the inlet 314 to progress or swim along a fluidic path toward the filter chamber 316. In still another illustrative embodiment shown in FIG. 5, the system 510 includes a pre-filter zone 530 includes an introduction channel 520 extending from an inlet 514 to a filter chamber 516, so as to allow sperm delivered to the fluidic system through the inlet 514 to progress or swim along a fluidic path toward the filter chamber 516. In another illustrative embodiment shown in FIG. 6, the system 610 includes a pre-filter zone 630 includes an introduction channel 620 extending from an inlet 614 to a filter chamber 616, so as to allow sperm delivered to the fluidic system through the inlet 614 to progress or swim along a fluidic path toward the filter chamber 616. In yet another illustrative embodiment shown in FIG. 7, the system 710 includes a pre-filter zone 730 includes an introduction channel 720 extending from an inlet 714 to a filter chamber 716, so as to allow sperm delivered to the fluidic system through the inlet 714 to progress or swim along a fluidic path toward the filter chamber 716.

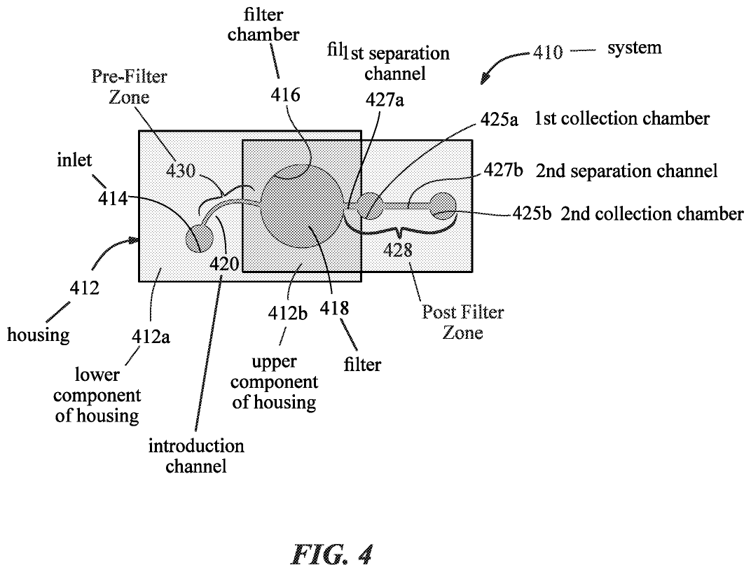

In other illustrative embodiments, such as is shown in FIG. 4, the system 410 includes a pre-filter zone 430 that is configured and/or adapted to sort sperm based on their swimming ability and is other than a single straight channel. The pre-filter zone 430 typically includes at least one introduction channel 420 that extends from the inlet 414 to the filter chamber 416, so as to allow sperm delivered to the fluidic system through the inlet 414 to progress or swim along a fluidic path toward the filter chamber 416. For example, in some illustrative embodiments, at least a portion of the introduction channel 420 is curved, such as shown in FIG. 4. In some exemplary embodiments, one, two or more portions of the introduction channel 420 are curved. In other exemplary embodiments, the entire introduction channel 420 is curved. The curvature of the introduction channel 420 (i.e., one or more portions thereof, or the entire introduction channel 420) is optimized for sorting the sperm introduced into the inlet 414, based upon their swimming ability. In some illustrative embodiments, the curvature, or bend, is between about 10-degrees on the low end to about 160-degrees on the high end. In some illustrative embodiments, the curvature is between 30-degrees and 150-degrees. In still other illustrative embodiments, the curvature is between 45-degrees and 135-degrees. In certain illustrative embodiments, the curvature is between 25-degrees and 75-degrees. For example, the curvature can be 30, 40, 45, 50 or 60 degrees. In other illustrative embodiments, the curvature is between 120-degrees and 150-degrees. In certain illustrative embodiments, the curvature is between 125-degrees and 145-degrees. For example, the curvature can be 125, 130, 135 or 140 degrees. In certain illustrative embodiments, the introduction channel 420 includes one or more structures adapted for enhancing the sorting of the sperm, such as but not limited to dams, pillars and the like. Accordingly, in some exemplary embodiments, the introduction channel 420 is configured and/or adapted to sort sperm based on their swimming ability, which is an indicator of the sperm's health, and is other than a single straight channel. In various illustrative embodiment, a curved introduction channel 420 (or pre-filter zone 430) can be combined with one or more post-filter zone configurations discussed herein. Various combinations of pre-filter zones 130 and post-filter zones 128 provided herein form certain illustrative embodiments.

In certain illustrative embodiments of a system 110 herein, some or all of the pre-filter zone 130 is optional, and therefore lacks all or any of the components of the pre-filter zone 130, including an inlet 114 and/or an introduction channel 120. Accordingly, in some exemplary embodiments, the lower portion 124 of the filter chamber 116 is accessed directly for deposition of a semen sample (e.g., the sample of sperm) therein. For example, in some illustrative embodiments, an orifice in the side of the housing lower component 112a is sized such that the tip of a pipette can be inserted through the wall of the housing lower component 112a and into the filter chamber lower portion 124, so that the sample comprising the sperm can be pipetted directly into the lower portion 124. For example, in an illustrative embodiment, the filter chamber upper and lower portions 126, 124 are at least partially filled with a liquid media, a semen sample is delivered into the lower portion 124 after which the orifice is optionally sealed, and the filter chamber can be filled. During an incubation period, some of the motile sperm swim up, through the micropores of the filter 118, and into the filter chamber upper portion 126. In a further illustrative embodiment, some of the motile sperm in the filter chamber upper portion 126 swim into the post-filter zone 128, such as is disclosed elsewhere herein. Any of the post filter zone 128 configurations disclosed herein can be used in such illustrative embodiments.

In some illustrative embodiments, the housing lower and upper components 112 and 112b, respectively, are configured and/or arranged to be separated, such that the semen sample can be pipetted directly into the lower portion 124 of the filter chamber 116, followed by reassembling the system 110, such that the upper portion 126 is again aligned over the lower portion 124, with the filter 118 therebetween. For example, in an illustrative embodiment, the upper component 112b and the filter 118 are disconnected from the lower component 112a, and liquid media is added to the lower portion 124 of the filter chamber 116. The semen sample is then pipetted into the media within the lower portion 124. Then the filter 118 and the upper component 112b are reconnected to the lower component 112a, such that the filter 118 and the upper portion 126 of the filter chamber 116 are vertically aligned with the lower portion 124 of the filter chamber 116, such that the filter chamber 116 is correctly reassembled. Media is added to the filter chamber upper portion 126 and the post filter zone 128, and the system 110 is incubated. During incubation, without the addition of an external force, some of the motile sperm in the semen sample self-sort themselves by swimming up through the micropores of the filter 118 and into the filter chamber upper portion 126. Then, some of the motile sperm within the filter chamber upper portion 126 swim into the post filter zone 128 to become further self-sorted. Any of the post filter zone 128 configurations disclosed herein can be used in such illustrative embodiments.

In some illustrative embodiments, a system herein comprises a reservoir formed within the lower component (212a) that is configured and arranged for receipt of a sperm sample (e.g., semen sample) therein. For example, the semen sample is placed in the reservoir under the lower portion 124, and such reservoir in some embodiments becomes part of the lower portion 124 when the system 110 is assembled together after the sample is added to the reservoir and before an incubation period during which motile sperm swim through the filter 118. After the motile sperm swim up through the filter 118, they can be withdrawn, harvested and/or collected through an opening in the filter chamber upper portion 126, if such an opening is present, or in illustrative embodiments they are harvested from a channel or chamber in any of the post filter zone 128 configurations disclosed herein.

### Post-Filter Zone

In illustrative embodiments, the system includes a post-filter zone that is typically configured and/or adapted to sort sperm based on their swimming ability, which can reflect the sperm's health, and which is other than a single straight separation channel connected to a single collection chamber and in some embodiments other than a single straight channel. In general, the post-filter zone includes at least two separation channels fluidly connected to two collection chambers, in various configurations discussed below. For example, the geometry (e.g., length, width, cross-sectional shape, curvatures, etc.) of the post-filter zone can be adapted to enhance sperm sorting. In another example, the post-filter zone can include sorting enhancing structures, such as but not limited to dams, pillars and the like, such as is discussed above with respect to the pre-filter zone. For example, any separation channel can include one or more, and typically a plurality or set of pillars of various shapes (e.g. cylindrical, square, rectangular), sizes (e.g. between 1/10 on the low end of the range and ⅕, ¼, ⅓, ½, ⅔, ¾, and ⅘ on the high end of the range, of the diameter, area, and/or width of a channel, or between ⅕ on the low end of the range and ¼, ⅓, ½, ⅔, ¾, and ⅘ on the high end of the range, of the diameter, area, and/or width of a channel) and spacing (e.g. between 100 μm on the low end of the range and 250 μm, 500 μm, 1 mm, 2 mm, and 5 mm on the high end of the range between the center of pillars in the channel, or between 250 μm on the low end of the range and 500 μm, 1 mm, 2 mm, and 5 mm on the high end of the range between the center of pillars in the channel), configured to, effective for, and adapted to modify the path of the sperm as they swim through the channel and thereby help to separate the more motile sperm and/or morphologically normal sperm from less motile and/or morphologically abnormal sperm as they swim toward a collection chamber.

As discussed above, the system can include a dam to facilitate separation of the more motile sperm, morphologically normal sperm from the less motile, morphologically abnormal sperm. In some illustrative embodiments, the system includes a dam that is present in a collection chamber and/or a separation channel so as to block sperm from traveling (i.e., swimming) past the point of the dam. Advantageously, in certain illustrative embodiments, the more motile sperm become concentrated in front of the dam. For example, in some illustrative embodiments, the sperm are concentrated at the collection chamber before the dam. In another illustrative embodiment, a dam is present at the junction of the first collection chamber and the second separation channel, or in the second separation channel to concentrate sperm in the first collection chamber.

In some illustrative embodiments, the post filter zone 128 includes one, and in illustrative embodiments two or more separation channels (e.g., such as is shown in FIG. 2, separation channel 227a and/or 227b) with an outlet formed by its open distal end. In such embodiments, some of the motile sperm can be withdrawn from the separation channel 227a or 227b by inserting a pipette tip into the open outlet and optionally into the channel instead of a collection chamber, and withdrawing or otherwise aspirating some of the motile sperm therefrom. For example, in a further illustrative embodiment, the post filter zone 128 comprises two or more fluidly connected channels or portions (e.g., as shown in FIG. 1B, separation channels 127a and 127b) with two or more open distal ends that form outlets, wherein the outlets are sized, configured and/or arranged for inserting a pipette tip therein, for withdrawing some of the motile sperm therefrom. Accordingly, in some embodiments there are outlets in one or more of the channels in the post filter zone 128 that are openings but not separate chambers, that are sized, configured and/or arranged for inserting a pipette therein, from which sperm can be withdrawn. In some embodiments, sperm can be withdrawn using such outlets from other channels or chambers that are fluidly connected to the channel comprising such outlets, and even from above the filter of the filter chamber.

Thus, the configuration of the post-filter zone is optimized for sperm sorting based upon the ability of the sperm to swim, and therefore the health of the sperm, which thereby can be used in some embodiments to collect one, two or more samples of sperm that are more suitable for downstream detection, counting, analysis, and/or ART procedures than the sperm that were deposited in the inlet, regardless of whether the initial sperm sample has millions or 10s of millions of healthy sperm or just a few healthy sperm. In illustrative embodiments, some, most, virtually all, or all of the sperm are harvested from a collection chamber. In illustrative embodiments, between 5% and 10%, 20%, 25%, 50%, 75%, 80%, 90%, 95%, 99% or 100% of the sperm are harvested from a collection chamber.

### Two Separation Channels and Collection Chambers

In illustrative embodiments, systems provided herein include a post-filter zone that includes two or more (e.g., a plurality of) separation channels connected to respective collection chambers, wherein such post-filter zone is configured to optimize sperm separation based upon the swimming ability of sperm in the sample. For example, in the illustrative embodiment shown in FIGS. 1A-1B, the post-filter zone 128 includes a first separation channel 127 fluidly connected, joined or coupled to the upper portion 126 of the filter chamber 116. The first collection chamber 125a (or proximal collection chamber) and the second collection chamber 125b (or distal collection chamber) are typically fluidly connected to the upper portion 126 of the filter chamber 116 by a first separation channel 127a and a second separation channel 127b, respectively. In some illustrative embodiments, the first and second separation channels 127a, 127b and the first and second collection chambers 125a, 125b provide for additional sorting of the motile sperm moving, or swimming, through the system 110, as compared to the motile sperm that have moved through the filter 118 and into the upper portion 126 (e.g., denoted by white arrows 02), so as to facilitate harvesting of the most motile and healthiest sperm, which are more likely to successfully fertilize an ovum or egg.

Various components of the system are optimized such that interactions of the sperm with the system facilitate separation of the most motile and likely healthiest sperm from the sperm sample applied to the inlet, so as to facilitate harvesting the best of the best sperm, which are more likely to achieve successful fertilization. For example, in the illustrated embodiment of FIGS. 1A-1B, the first separation channel 127a is shorter than the second separation channel 127b.

The first separation channel 127a is configured to facilitate harvesting at least some of the motile sperm from the first collection chamber 125a and includes a length that is optimized to sort sperm based on their swimming ability, so that a greater number of more motile and likely healthier sperm reach the first collection chamber 125a, as opposed to less motile or less healthy sperm. The configuration of the first separation channel 127a, in illustrated embodiments, can include its size, shape, and relationship to other components of the system 110, such as for example, its length, cross-sectional shape, the presence of sperm chemo-attractants, its orientation relative to the upper portion 126 (i.e., being curved as shown in FIG. 4), and the like. In these embodiments, the first separation channel 127a is fluidly connected with the first collection chamber 125a, which is configured with an orifice that is configured and/or adapted such that the tip of a micropipette (or other sperm collection device) can be inserted therein, such that motile sperm within the first collection chamber 125a can be withdrawn or harvested. Thus, the first collection chamber 125a has an appropriate size, shape, configuration and relationship to the other components of the system 110 to facilitate harvesting of the sperm from the first collection chamber 125a.

In these embodiments, the second separation channel 127b extends through the upper component 112b and is fluidly connected to a second collection chamber 125b, which is configured with an orifice such that the tip of a micropipette can be inserted therein, such that motile sperm within the second collection chamber 125b can be withdrawn or harvested. In some embodiments, such as is shown in FIGS. 1A and 1B, the second separation channel 127b is fluidly connected to the first collection chamber 125a. In other embodiments, such as is shown in FIG. 2 or FIG. 7, the second separation channel (227b and 727b, respectively) is connected directly to the upper portion (226 and 726, respectively) of the filter chamber (216 and 716, respectively). The second collection chamber 125b is configured and/or adapted to facilitate harvesting at least some of the motile sperm from the second separation channel 127b, such as by inserting the tip of a micropipette into the second collection chamber 125b and withdrawing a sample of the motile sperm within the second collection chamber 125b. Similar to the first separation channel 127a, the second separation channel 127b is configured and/or adapted to sort sperm based on their swimming ability, so as to facilitate harvesting at least some of the more motile sperm from the second collection chamber 125b. For example, the second separation channel 127b includes a length that is optimized so that a greater number, concentration, or percentage, of more motile or healthier sperm reach the second collection chamber 125b, as opposed to less motile or less healthy sperm. The configuration of the second separation channel 127b, in illustrated embodiments, can include its size, shape, configuration and relationship to the other components of the system 110, such as for example, its length, cross-sectional shape, the presence of sperm chemo-attractants, its orientation relative to the upper portion 126 (i.e., it can be angled or curved relative to the filter chamber, such as is shown in FIGS. 2 and 7) and/or the first collection chamber 125a, and the like. Similar to the first collection chamber 125a, the second collection chamber 125b, is configured with an orifice that is configured and/or adapted such that the tip of a micropipette (or other sperm collection device) can be inserted therein, such that motile sperm within the second collection chamber 125b can be withdrawn or harvested. Thus, the second collection chamber 125b has an appropriate size, shape, configuration and relationship to the other components of the system 110 to facilitate harvesting of the sperm from the second collection chamber 125b. The system 110 can include additional collection chambers (i.e., 125c, 125d, 125e, etc., which can be referred to as the least, less, more or most proximal or distal, as would make sense to one skilled in the art), which also have an appropriate size, shape, configuration and relationship to the other components of the system 110 to facilitate harvesting of the sperm therefrom.

In certain illustrative embodiments, a fluid control subassembly is fluidly connected to the system 100, so as to provide a positive or negative pressure or force for at least a period of time during performance of a method herein. Thus, in such embodiments, an external force can be applied after sperm are delivered into a device to perform all or a portion of any of the steps provided in methods herein, such as for sorting sperm. In illustrative embodiments, the fluid control subassembly is fluidly connected to one or both of the pre-filter zone 130 and the post-filter zone 128. In an illustrative example, the fluid control subassembly is fluidly connected to the pre-filter zone 130 and configured, arranged, adapted and operational to apply a positive pressure, so as to push a sample of sperm added to the inlet 114 toward, and optionally into, the lower portion 124 of the filter chamber 116. For example, in some illustrative embodiments, the fluid control subassembly can apply positive pressure, to thereby push the sample of sperm forward through the introduction channel 120, until the sperm reach the lower portion 124, and then the fluid control subassembly turns off, such that, for example, the sperm in the lower portion 124 are then separated based upon their ability to swim through the filer 118 and into/through the post-filter zone 128. In another illustrative example, fluid control subassembly is fluidly connected to the post-filter zone 128, and configured, arranged, adapted and operational to apply a negative pressure to one or more of the collection chambers. In a further illustrative example, the fluid control subassembly intermittently applies the negative pressure to the one or more collection chamber after the sperm have reached the one or more collection chambers, such as but not limited to, for example, at the end of an incubation period, such as for example to withdraw, collect and/or harvest a sample of sperm therefrom. In some illustrative examples, the fluid control subassembly operates intermittently and/or periodically, and optionally is part of an automated system configured to sort and harvest sperm from the one or more collection chambers in the post-filter zone 128. In some illustrative embodiments, additional system configurations, such as but not limited to those shown in FIGS. 2-7, can be used in combination with such a fluid control subassembly.

In some embodiments, a computer processor can be used to control such fluid control subassembly. Such processor can be programmed using a software program loaded into a computer memory with instructions, for example, to perform any, most or all steps of any embodiment herein. For example, the fluid control subassembly can be instructed to start to harvest a portion or all of a sample in a collection chamber after a set period of time from the moment a sample is delivered into a system herein. Such set period of time can be any of the times provided herein in methods for sorting or otherwise separating sperm.

The lengths of the first and second separation channels 127a and 127b typically are selected so as to facilitate sorting sperm based upon their swimming ability, so as to facilitate collection of healthier, more motile sperm. Furthermore, the lengths of these separation channels and their configurations with respect to the upper portion 126 of the filter chamber 116, to which they are fluidly connected, can be selected so as to facilitate additional functionality of the present systems and methods. For example, in some embodiments, systems and methods herein can be used to collect/harvest two samples of motile sperm. One sample can be collected/harvested from the first collection chamber 125a and a second sample from the second collection chamber 125b. In illustrative embodiments, the first collection chamber 125a can be used to collect more sperm, for example for artificial insemination procedure(s), and the second collection chamber 125b can be used to collect fewer sperm, for example for in vitro fertilization procedure(s). Thus, in some embodiments between 10 and 1,000,000 times less, or between 1,000 and 1,000,000 times less sperm, are contained in the second withdrawn sample than the first withdrawn sample.

In embodiments, such as those discussed in the paragraph above, the first collection chamber 125a, which in such embodiments can be present or located before the second collection chamber 125b in the fluidic path, can be used to collect hundreds, thousands, tens of thousands, hundreds of thousands, or millions of sperm cells in the first withdrawn or harvested sample. Accordingly, methods herein that for example utilize a system 110 herein that includes such two collection chamber configurations can be used to harvest 1 to 10,000,000, or in illustrative embodiments, 100,000 to 2,000,000 sperm cells. In some illustrative embodiments, 100 sperm cells on the low end of the range, to 1,000 sperm cells on the high end of the range can be harvested. In some illustrative embodiments, 1,000 sperm cells on the low end of the range, to 10,000 sperm cells on the high end of the range can be harvested. In some illustrative embodiments, 10,000 sperm cells on the low end of the range, to 100,000 sperm cells on the high end of the range can be harvested. In some illustrative embodiments, 100,000 sperm cells on the low end of the range, to 1,000,000 sperm cells on the high end of the range can be harvested. In some illustrative embodiments, 50,000 sperm cells on the low end of the range, to 100,000,000 sperm cells on the high end of the range can be harvested. Such a withdrawn, collected or harvested sample with the relatively large numbers of motile sperm can be particularly well adapted and useful for an artificial insemination procedure (i.e., AI). In such embodiments, the second collection chamber 125b can be used to collect between 1 and 100 sperm cells, or between 1 and 10 sperm cells in the second withdrawn sample. In some illustrative embodiments, 1 sperm cell on the low end of the range, to 20 sperm cells on the high end of the range can be harvested. In some illustrative embodiments, 10 sperm cells on the low end of the range, to 50 sperm cells on the high end of the range can be harvested. In some illustrative embodiments, 15 sperm cells on the low end of the range, to 200 sperm cells on the high end of the range can be harvested. As a non-limiting example, sperm cells harvested from the second withdrawn sample can be used in a procedure that utilizes and/or requires 10 sperm cells or fewer, or typically utilizes and/or requires only one sperm cell, such as an intracytoplasmic injection (i.e., ICSI).

In a further illustrative embodiment, at least some of the motile sperm harvested from the first collection chamber 125a and the second collection chamber 125b can be compared so as to determine the most motile or otherwise most fit for fertilization. For example, the motility and/or the morphology of the sperm from the first and second collection chambers 125a, 125b can be measured, using techniques known in the art, and then compared, so as to evaluate them. In this manner, the user can select the sperm most suitable for an in vivo or in vitro procedure. For example, suppose the first collection chamber 125a contains 100,000 sperm cells and the second collection chamber 125b contains only 1,000 sperm cells, the practitioner may elect to perform IUI with the sperm harvested from the first collection chamber 125a; the sperm cells from the second collection chamber 125b can be frozen for use in an ICSI procedure at a later date, as a backup. In another example, suppose the first collection chamber 125a contains 10,000,000 sperm cells and the second collection chamber 125b contains 80,000 sperm cells, the practitioner may elect to perform IUI with the sperm harvested from the second chamber 125b, as store the sperm harvested from the first collection chamber 125a as a backup for later IUI attempts.

In some circumstances, based upon the evaluation, the user may decide to preserve one or both samples of harvested sperm. In other circumstances, based upon the evaluation, the user can advise the patient to return at a later time point for additional sperm collection. In some circumstances, such as but not limited to an insufficient number of motile sperm being harvested, such as but not limited to the collected sperm sample being of poor quality, the user can pool multiple samples of motile sperm harvested from one or both collection chambers 125a, 125b, including with previously preserved harvested samples.

In certain illustrative embodiments, one or both of the collection chambers 125a, 125b is an outlet or orifice configured and/or arranged for the insertion of the tip of a pipette therein, for the withdrawal, collection and/or harvesting of a sample of the motile sperm from the associated separation channel 127a, 127b. In other illustrative embodiments, one or both of the collection chambers 125a, 125b is a chamber configured and/or arranged for the insertion of the tip of a pipette therein, for the withdrawal, collection and/or harvesting of a sample of the motile sperm therefrom, typically by having an opening usually at the top of the collection chambers 125a, 125b, large enough for the pipette or tip thereof.

### Sets of Collection Chambers and Separation Channels

In further embodiments, the post-filter zone includes additional collection chambers, such as but not limited to a 3rd, 4th, or more collection chambers, such as but not limited to a plurality or set of collection chambers. For example, in the illustrative embodiment shown in FIG. 5, the post-filter zone 528 includes a series of collection chambers, for example a series of five collection chambers (i.e., 525a, 525b, 525c, 525d, 525e) fluidly connected by a series of separation channels, for example a series of five separation channels (i.e., 527a, 527b, 527c, 527d, 527e), organized in a serial configuration. In another illustrative embodiment, as shown in FIG. 3, instead of a single second separation channel, the post-filter zone 328 includes a plurality of parallel separations channels (i.e., 327b, 327c, 327d, 327e and 327f) fluidly joining the first collection chamber 325a with the second separation chamber 325b. In yet another illustrative embodiment, shown in FIG. 6, the post filter zone 628 includes a first collection chamber 625a fluidly joined or connected by a first separation channel 627a with the filter chamber 618, and two or more (e.g. a plurality of) additional collection chambers (i.e., 625b, 625c, 625d, etc.) fluidly joined to a first collection chamber 625a by respective separation channels (i.e., 627b, 627c, 627d, etc.) such as but not limited to in a flower or spokes of a wheel configuration. Various embodiments include fewer or more collection chambers fluidly connected with the first collection chamber 625a. The separations channels (627a, 627b, 627c, 627d, etc.) can be of equal or non-equal lengths. In illustrative embodiment, the post-filter zone 128 can include a set or plurality of collection chambers (i.e., 125x, wherein x is any letter designating a specific or given collection chamber in the set of collection chambers) and associated separation channels, such as but not limited to 2, 3, 4, 5, 6, 7, 8, 9, 19, 11, 12, 20, or 25 collection chambers, each of which includes an associated, respective separation channel (i.e., 127y, wherein y is any letter designating a specific or given separation channel associated with a particular collection chamber 125x in the set thereof).

In an illustrative embodiment, the post filter zone comprises a plurality or a set of separation channels (i.e., 127a, 127b, 127c, etc.) comprising a first separation channel 127a that is directly connecting to the upper portion 126 and to a first collection chamber 125a, a second separation channel 127b, and a third separation channel, wherein each separation channel of the set of separation channels, other than the first separation channel is connected to two collection chambers of a plurality or set of collection chambers (i.e., 125a, 125b, 125c, etc.) comprising the first collection chamber, a second collection chamber and a third collection chamber, wherein collection chambers of the plurality or set of collection chambers are configured to facilitate harvesting some or all of the motile sperm therein. In illustrative embodiments, the sum of the lengths of the three or more separation channels is between 15 mm and 100 mm.

In some illustrative embodiments, the plurality or set of separation channels comprises at least five separation channels and the plurality or set of collection chambers comprises at least five collection chambers. In some illustrative embodiments, the plurality or set of separation channels have equal lengths. In some illustrative embodiments, at least one separation channel of the plurality or set of separation channels has a different length than the other separation channel(s) of the set. For example, one separation channel can have a first length and the remaining four separation channels can have a second length. In another example, all of the separation channels can have different lengths.

### Parallel Separation Channels

In some illustrative embodiments, the set of separation channels includes two or more parallel separation channels. In some illustrative embodiments, the set of separation channels comprises a set of parallel channels, such as but not limited to as shown in FIG. 3, which includes five parallel separation channels 327b, 327c, 327d, 327e and 327f. In some illustrative embodiments, the set of parallel channels comprise a single inlet and/or a single outlet. For example, as shown in FIG. 3, the five parallel separation channels 327b, 327c, 327d, 327e and 327f are fluidly connected with the first collection chamber 325a and the second collection chamber 325b.

### Curved Separation Channels

In yet another illustrative embodiment, a portion or all of one or more of the separation channels can include a curvature or bend that is optimized for sorting sperm based upon their swimming ability, in a manner similar to that discussed above with respect to the introduction channel. In some illustrative embodiments, the curvature, or bend, of at least a portion of one or more of the separation channels is between about 10-degrees on the low end to about 160-degrees on the high end. In some illustrative embodiments, the curvature is between 30-degrees and 150-degrees. In still other illustrative embodiments, the curvature is between 45-degrees and 135-degrees. In certain illustrative embodiments, the curvature is between 25-degrees and 75-degrees. For example, the curvature can be 30, 40, 45, 50 or 60 degrees. In other illustrative embodiments, the curvature is between 120-degrees and 150-degrees. In certain illustrative embodiments, the curvature is between 125-degrees and 145-degrees. For example, the curvature can be 125, 130, 135 or 140 degrees. As the sperm swim through the separation channels, they interact with the walls thereof, which promotes separation of the more fit sperm from the less fit sperm. Additional configurations of the post-filter zone are foreseen.

In an illustrative embodiment, the introduction channel (e.g., 420 in FIG. 4) and/or the first separation channel and/or second separation channel (e.g., the second separation channel 727b in FIG. 7) is curved or comprises a curved section; and/or the introduction channel and/or the first separation channel and/or the second separation channel comprises one or more 45 to 135 degree bends or curves, such as are described above.

### Dimensions and Layers

Systems and devices herein, in illustrative embodiments (See e.g., FIG. 1 A), have a rectangular shape. For example, systems and devices herein can have a shape such that the length is between 1.1× and 10.0×, 1.2× and 5×, 1.2× and 2.5×, 1.2× and 2×, 1.2× and 2.2×, or 1.2× and 2.4× longer than the width. Exemplary dimensions for example, include a width of between 1.5 cm and 5 cm and a length between 3 cm and 15 centimeters. In some embodiments, the width of the device can range in width from 1.5 cm to 10 cm, for example, from 1.5 cm to 7.5 cm, from 1.5 to 5.0 cm, from 1.5 cm to 4.5 cm, from 1.5 cm to 4.0 cm, from 1.5 cm to 3.5 cm, or from 1.5 to 3.0 cm. In some embodiments, the length of the device can range in size from 3 cm to 15 cm in length, for example, from 3 cm to 12 cm in length, from 3 cm to 10 cm, from 3 cm to 8 cm, from 3 cm to 6 cm in length. In some embodiments, the length can range from 3 cm to 15 cm, for example, from 3 cm to 15 cm, from 5 cm to 15 cm, from 7 cm to 15 cm, from 9 cm to 15 cm, from 11 cm to 15 cm, or from 12 cm to 15 cm in length. In illustrative embodiments, the system or device has a rectangular shape, and thus the length is greater than the width, in some embodiments the length is 1.1 to 2, 1.75, 1.5, 1.25, or 1.1 times greater than the width. In some embodiments, the upper component is directly above the lower component. In other embodiments, for example as illustrated in FIG. 1, the upper component is offset from the lower component, for example such that at least 10, 15, 20, 25, 30, 40, 50, 60, or 75% of the upper component is not directly above the lower component. In some embodiments, less than 75, 60, 50, 40, 30, 25, 20, 10, or 5% of the upper component is not directly above the lower component. In some embodiments, substantially all or all of the upper component is directly above the lower component.

As disclosed hereinabove, the lower component in certain illustrative embodiments is a lower layer, and the upper component in certain illustrative embodiments, is an upper layer. Although in some embodiments only one of the lower component and the upper component is configured as a layer, in illustrative embodiments, the lower component is a lower layer and the upper component is an upper layer. In such a configuration where one or more of the components are layers, the components have a relatively thin and flat structure, for example such that the component is not more than 25, 20, 15, 10, 5, or 2 mm thick, for example between 2 and 25 mm, 2 and 20 mm, 2 and 15 mm, 3 and 12 mm, or 4 and 10 mm thick. The lower layer and the upper layer can either or both have a substantially flat, or a flat upper and/or lower surface. A substantially flat surface can have, for example a circular ridge around a port, or can have a ledge around the perimeter of a cavity that forms a filter chamber that can be used to affix the edges of a filter thereto.

In some embodiments, components, including in some embodiments, component layers, disclosed herein can be connected, affixed, attached, and/or coupled together using a chemical bond or using an adhesive. Components that are attached or connected together by a chemical bond or an adhesive, are typically not detached by an end user during intended use of the device that includes such components. It will be understood that many different chemical bonds and adhesives are known and can be used to connect the components in systems and devices herein, including illustrative systems and devices wherein the components are composed of plastic. For example, the adhesive can be a silicone adhesive or the adhesive found on a commercially available double sided strip with adhesive on both sides (DSA), such as double-sided tape. As disclosed herein in certain illustrative embodiments, the lower component is adhered to a substrate with DSA. This can provide an advantage of forming a channel and connecting the lower component and the substrate, as exemplified herein. Furthermore, the upper component and the lower component in an illustrative embodiment are coupled together with a DSA framed membrane filter subassembly wherein the DSA can be attached to the filter, or to a plastic frame housing the filter in the filter sub-assembly.

The diameters and widths of channels are typically smaller than the diameters of the filter chamber in the device, as illustrated in FIG. 1A and FIG. 1B for example. In some embodiments, some channels and chambers have diameters in micrometers or less, such as 1 to 999 micrometers. In other embodiments, all channels in the device are less than 1 mm in diameter and all chambers are 1 mm or larger in diameter.

In some embodiments, some channels and chambers have diameters in micrometers or less, such as 1 to 999 micrometers, and some channels and chambers have dimensions that are least 1 mm in diameter (e.g. 1 mm to 10, 100, or 1000 mm). In some embodiments, all channels in the device are less than 1 mm in diameter and all chambers are 1 mm or larger in diameter in diameter.

In some embodiments, the diameter of the filter chamber is between 5 mm on the low end of the range and 100 mm, 75 mm, 50 mm, 40 mm, 30 mm, 25 mm, and 20 mm on the high end of the range, or between 5 mm, 10 mm, 15 mm, 20 mm, 25 mm, or 30 mm on the low end of the range, and 100 mm on the high end of the range, or between 10 mm on the low end of the range and 100 mm, 75 mm, 50 mm, 40 mm, 30 mm, 25 mm, and 20 mm on the high end of the range, or between 5 mm, 10 mm, 15 mm, 20 mm, 25 mm, or 30 mm on the low end of the range, and 75 mm on the high end of the range, or between 15 mm and 50 mm, or between 20 mm and 30 mm, or between 30 mm and 50 mm, or between 30 mm and 40 mm, or between 20 and 25 mm.

The lengths of any of the channels herein, can be the lengths disclosed herein for the first separation channel or the second separation channel with respect to FIGS. 1A, 1B, and 3. The inlet, channels and chambers of systems and devices herein in cross section, can be round, square, triangular, and in non-limiting, illustrative examples are circular. In some embodiments, the diameter, or width, of the inlet, channels and collection chamber(s) is between 0.5 mm on the low end of the range and 15 mm, 12.5 mm, 10 mm, 7.5 mm, 5.0 mm, 2.5 mm, or 2.0 mm on the high end of the range, or between 0.5 mm, 1.0 mm, 1.5 mm, 2.0 mm, 2.5 mm, 3.0 mm, 3.5 mm, 4.0 mm, 4.5 mm, or 5 mm on the low end of the range, and 15 mm on the high end of the range, or between 2 mm, 2.5 mm, 3.0 mm, 3.5 mm, 4.0 mm, 4.5 mm, 5 mm, 5.5 mm, 6.0 mm, on the low end of the range and 10 mm on the high end of the range, or between 12 mm. 10 mm, 8 mm, 6 mm, or 4 mm on the high end of the range, and 2.0 on the low end of the range, or between 1.5 mm and 8 mm, or between 2 mm and 10 mm, or between 2.5 mm and 8 mm, or between 3 mm and 7 mm, or between 4 mm and 10 mm, or between 6 mm and 12 mm.

The inlet, (also referred to as inlet port) can range from 1.5 mm to 6 mm in diameter, for example, from between 1.0 mm to 5.5 mm in diameter, from 1.0 to 5.0 mm in diameter, from 1.0 mm to 4.5 mm in diameter, or from 1.0 to 4.0 mm in diameter. In some embodiments, the inlet can range from 1.0 mm to 6 mm in diameter, from 1.5 mm to 6 mm in diameter, from 2.0 mm to 6 mm in diameter, or from 3.5 to 6 mm in diameter. In non-limiting illustrative embodiments, the width of the inlet can range from 1.5 mm to 4.5 mm in diameter. In other illustrative embodiments, the inlet can range from 1.5 mm to 4.0 mm in diameter. In the inlet can be cut into the lower component at a depth of from between 2.0 mm to 9 mm deep, for example, from between 2.0 mm to 8.5 mm, from 2.0 mm to 8.0 mm, from 2.0 mm to 7.5 mm, from 2.0 mm to 7.0 mm, from 2.0 mm to 6.5 mm, from 2.0 mm to 6.0 mm, from 2.0 mm to 5.5 mm, from 2.0 mm to 5.0 mm, from 2.0 mm to 4.5 mm, from 2.0 mm to 4.0 mm, from 2.0 mm to 3.5 mm, or from 2.0 mm to 3 mm deep. In some embodiments, the inlet depth can range from between 2.0 mm to 9 mm deep, for example, from 2.5 mm to 9 mm, from 3.0 mm to 9 mm, from 3.5 mm to 9 mm, from 4.0 mm to 9.0 mm, from 4.5 mm to 9.0 mm, from 5.0 mm to 9 mm, from 5.5 mm to 9 mm, from 6.0 mm to 9 mm, from 6.5 mm to 9 mm, from 7.0 mm to 9 mm or from 7.5 mm to 9 mm, on the high end. In some embodiments, the dimensions of the inlet are compatible to the dimensions of a distal section of a needleless syringe, so that the syringe can be inserted into the inlet port and held in a vertical position. In some embodiments, a distal portion of a syringe can be inserted into the inlet so that there is a seal (i.e., no air gap). In some embodiments, a distal portion of a syringe can be inserted in the inlet so that there is an air gap, and/or a syringe or pipet is not held in place in a vertical position. In some embodiments, the system 110 is configured to, effective for, adapted to, and capable of receiving and/or holding a liquid sample (e.g., semen) comprising sperm (cither fresh or frozen, processed or raw) that has a volume ranging from 50 μl at the low end of the range and 250 μl, 500 μl, 750 μl, 1 ml, 2 ml and 5 ml at the high end of the range. For example, in embodiments, devices herein can receive (i.e., hold) a volume of fluid without causing membrane expansion due to overfilling, or without overflowing. For example, in non-limiting embodiments, the sample volume that can be loaded in the device, held in the lower portion, and/or held in the upper portion of the filter chamber is between 0.25 ml on the low end of the range and 25 ml, 20 ml, 10 ml, 7.5 ml, 6.5 ml, 6.0 ml, 5.5 ml, 5.0 ml, 4.5 ml, 4.0 ml, 3.5 ml, 3.0 ml on the high end of the range, or between 0.25 ml, 0.5 ml, 0.75 ml, 1.0 ml, 1.25 ml, 1.50 ml, 1.75 ml, 2.0 ml, or 2.5 ml on the low end of the range, and 25 ml on the high end of the range, or between 0.75 ml on the low end of the range and 10.0 ml, 7.5 ml, 6.5 ml, 5.5 ml, 4.5 ml, 3.5 ml, 2.5 ml, and 1.5 ml on the high end of the range, or between 0.5 ml, 1.0 ml, 1.5 ml, 2.0 ml, or 2.5 ml on the low end of the range, and 4.0 on the high end of the range, or between 0.5 ml and 5 ml, or between 0.75 and 3 ml, or between 0.75 ml and 1.75 ml, or between 1 ml and 3 ml, or between 1.5 ml and 3 ml, or between 2.0 ml and 3.5 ml. In some embodiments, the volume of the upper portion is between 40%, 50%, 60% 70%, 80%, 90% or 100% of the volume held in the lower portion.

### Separation Channel Lengths

The lengths of the separation channels and illustrative embodiments introductions channels are optimized for separating sperm based on their swimming ability, so that the more motile sperm can be harvested. In illustrative embodiments, the length of the first separation channel (e.g., first separation channel 127a in FIGS. 1A-1B, first separation channel 227a in FIG. 2, first separation channel 327a in FIG. 3, etc.) includes a length that optimizes selection of healthy, more motile sperm. For example, in some illustrative embodiments, the first separation channel has a length of 0.1 mm on the low end of the range to 100 mm on the high end of the range. In some illustrative embodiments, the first separation channel includes a length of 0.5 mm to 30 mm, 1 mm to 5 mm, 1 mm to 20 mm, 1 mm to 60 mm, or 0.5 mm to 30 mm. In some illustrative embodiments, the first separation channel includes a length of 1 mm, 2 mm, 3 mm, 5 mm, 7 mm, 10 mm, 15 mm, 20 mm, 25 mm, 30 mm, or 35 mm. In some illustrative embodiments, the first separation channel has a length of less than 1 mm or greater than 50 mm.

In illustrative embodiments, the length of the second separation channel (e.g., second separation channel 127b in FIGS. 1A-1B, second separation channel 227b in FIG. 2, second separation channel 327b in FIG. 3, etc.) includes a length that optimizes selection of healthy, more motile sperm. For example, in some illustrative embodiments, the second separation channel has a length of 0.1 mm on the low end of the range to 100 mm on the high end of the range. In some illustrative embodiments, the second separation channel includes a length of 0.5 mm to 30 mm, 1 mm to 5 mm, 1 mm to 20 mm, 5 mm to 20 mm, 0.5 mm to 30 mm, or 1 mm to 60 mm. In some illustrative embodiments, the second separation channel includes a length of 1 mm, 2 mm, 3 mm, 5 mm, 7 mm, 10 mm, 15 mm, 20 mm, 25 mm, 30 mm, or 35 mm. In some illustrative embodiments, the second separation channel 127b has a length of less than 1 mm or greater than 50 mm.

In some embodiments, the length of the first separation channel (e.g., first separation channel 127a in FIGS. 1A-1B, first separation channel 227a in FIG. 2, first separation channel 327a in FIG. 3, etc.) is equal to the length of the second separation channel (e.g., second separation channel 127b in FIGS. 1A-1B, second separation channel 227b in FIG. 2, second separation channel 327b in FIG. 3, etc.). In other embodiments, the first separation channel (e.g., first separation channel 127a in FIGS. 1A-1B, first separation channel 227a in FIG. 2, first separation channel 327a in FIG. 3, etc.) is longer than the second separation channel (e.g., second separation channel 127b in FIGS. 1A-1B, second separation channel 227b in FIG. 2, second separation channel 327b in FIG. 3, etc.). For example, in some illustrative embodiments, the length of the first separation channel is at least 0.25, 0.5, 0.75, 1.0, 1.25, 1.5, 1.75, or 2.0-times the length of the second separation channel. In some embodiments, the length of the first separation channel is more than 2 times the length of the second separation channel. In still other embodiments, the length of the second separation channel is greater than the length of the first separation channel. For example, in some embodiment, the length of the second separation channel is at least 0.25, 0.5, 0.75, 1.0, 1.25 or 1.5-times the length of the first separation channel. In some embodiments, the length of the second separation channel is between 1.5-times and 10-times the length of the first separation channel. For example, in certain embodiments, the length of the second separation channel is about 1.5, 1.75, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.5, 9.75 or 10-times the length of the first separation channel.

In some illustrative embodiments, the first separation channel (e.g., first separation channel 127a in FIGS. 1A-1B, first separation channel 227a in FIG. 2, first separation channel 327a in FIG. 3, etc.) has a length of less than 5 mm, the second separation channel (e.g., second separation channel 127b in FIGS. 1A-1B, second separation channel 227b in FIG. 2, second separation channel 327b in FIG. 3, etc.) has a length of between 5 and 20 mm, and the length of the second separation channel (e.g., second separation channel 127b in FIGS. 1A-1B, second separation channel 227b in FIG. 2, second separation channel 327b in FIG. 3, etc.) is at least 1.5 times the length of the first separation channel (e.g., first separation channel 127a in FIGS. 1A-1B, first separation channel 227a in FIG. 2, first separation channel 327a in FIG. 3, etc.).

### Separation Channel Widths

The separation channels (e.g., first separation channel 127a and second separation channel 127b in FIGS. 1A-1B, first separation channel 227a and second separation channel 227b in FIG. 2, first separation channel 327a and second separation channel 327b in FIG. 3, etc.) include a cross-section with a width. In some illustrated exemplary embodiments, the widths of one or both of the separation channels (e.g., separation channels 127a and 127b) can be constant along the length thereof. In some exemplary embodiments, the width of the first separation channel (e.g., separation channel 127a) can be equal to the width of the second separation channel (e.g., separation channel 127b). For example, both the first and second separation channels e.g., separation channels 127a and 127b) can be 1.5 mm wide along its length, respectively. In some exemplary embodiments, the width of the first separation channel (e.g., separation channel 127a) can be smaller than the width of the second separation channel (e.g., separation channel 127b). For example, the first separation channel (e.g., separation channel 127a) can be 1 mm wide along its length, while the second separation channel (e.g., separation channel 127b) can be 2 mm wide along its length. In some exemplary embodiments, the width of the first separation channel (e.g., separation channel 127a) can be greater than the width of the second separation channel (e.g., separation channel 127b). In another example, the widths of both the first and second separation channels (e.g., separation channels 127a and 127b) can be 1.5 mm along their lengths. For example, the first separation channel (e.g., separation channel 127a) can be 2 mm wide along its length, while the second separation channel (e.g., separation channel 127b) can be 1 mm wide along its length. In some exemplary embodiments, the width of one or both of the first and second separations channels (e.g., separation channels 127a and 127b) can vary along the length thereof. For example, the width can increase or decrease in size as sperm progress along the length of the particular respective separation channel. For example, the width of a channel cross-section can increase from 0.5 mm to 2 mm from one end of the separation channel to the other end thereof. Or for example, the width of a channel cross-section can decrease from 2 mm to 0.5 mm from one end of the separation channel to the other end thereof. Alternatively, the change in width can alternate between increasing and decreasing portions along the length of a separation channel (i.e., alternate one or more times along the length of the separation channel). If the system (e.g., systems 510 and 610) includes additional separation channels, such as shown in FIG. 5 and FIG. 6, for example, the additional separation channels (e.g., separation channels 527c, 527d, 527e, 627c, 627d, 627e, etc.) can also have a constant width (equal or different) and/or can have a variable width.

In some illustrative embodiments, the set of separation channels have equal lengths. In some illustrative embodiments, at least one separation channel of the set of separation channels has a different length than another separation channel of the set. In some illustrative embodiments, each of the separation channels in the set of separation channels has a length of between about 1 mm and 60 mm, and wherein the width of each of the separation channels is between 0.1 mm and 20 mm. In some illustrative embodiments, the first separation channel (e.g., separation channel 127a) has a length of between 1 to 5 mm. In some illustrative embodiments, the second separation channel (e.g., separation channel 127b) has a length of between about 10 mm and 60 mm. In some illustrative embodiments, the combined length of the separation channels in a sperm swim path in the fluidic system is between 25 mm and 100 mm. In some illustrative embodiments, the second separation channel (e.g., separation channel 127b) is between 1.5× and 25× the length of the first separation channel (e.g., separation channel 127a). In some illustrative embodiments, the length of any channel in the set is between 0.5× and 100× the length of any other channel in the set. In some illustrative embodiments, the second separation channel (e.g., separation channel 127b) is between 2× and 10× longer than the first separation channel (e.g., separation channel 127a). In some illustrative embodiments, the width of the first separation channel (e.g., separation channel 127a) is between 1 mm and 5 mm.

In some illustrative embodiments, a width of at least one of the first and second separation channel (e.g., separation channels 127a and 127b) includes a variable width, and wherein the variable width is one or more of progressively increasing, progressively decreasing, and alternating between increasing and decreasing. In some illustrative embodiments, the variable width comprises a width of 0.1 to 5 mm in narrowest width, and between 10 and 20 mm in largest width. In some illustrative embodiments, the variable width comprises a width of 0.1 to 2 mm in narrowest width, and between 10 and 20 mm in largest width. In some illustrative embodiments, the width of all of the channels in the fluidic path is between 1 mm and 10 mm.

In some illustrative embodiments, the fluidic system, such as for example the fluidic system 110 of FIGS. 1A-1B, is configured such that the motile sperm 01 move through the filter 118 into the upper portion 126 and then move from the upper portion 126 into the first collection chamber 125a without the application of an external force such that a percentage of motile sperm are present within the first collection chamber 125a that is greater than a percentage of motile sperm present within the lower portion 124. Accordingly, the motile sperm swim from the lower portion 124, up through the filter 118, include to the upper portion 126, through the first separation channel 127a and into the first collection chamber 125a. This sorting results in a greater percentage of the more motile being in the first collection chamber 125a than are in the lower portion 124, which can contain dead, dying and less motile sperm.

In some illustrative embodiments, the fluidic system 110 is configured such that the motile sperm 01 move through the filter 118 into the upper portion 126, and then move from the upper portion 126 into the first collection chamber 125a, and then move from the first collection chamber 125a into the second collection chamber 125b) without the application of an external force such that a percentage of motile sperm present within the second collection chamber 125b is greater than a percentage of motile sperm present in the sample.

In some illustrative embodiments, the fluidic system 110 is configured such that some of the motile sperm pass through the filter 118 into the upper portion 126 and move from the upper portion 126 into the second collection chamber 125b without the application of an external force.

In some illustrative embodiments, at least one of the first collection chamber 125a and the second collection chamber 125b are adapted for insertion of a pipette tip therein.

In some illustrative embodiments, the sperm are present in the inlet 114 and wherein the top end of the filter chamber is open to the atmosphere.

In some illustrative embodiments, the system 110 further comprises sperm within the at least one introduction channel 120 but not above the filter 118.

In some illustrative embodiments, the at least one introduction channel, the first collection chamber 125a and the second collection chamber 125b do not comprise a filter.

In some illustrative embodiments, the housing 112 comprises a support 122, wherein the support 122 is located under the lower component 112a and forms a lower surface of the at least one introduction channel 120.

In some illustrative embodiments, the filter 118 is a polycarbonate filter.

In some illustrative embodiments, no other channels that are open outlets or are connected to one or more open outlets are directly connected to the upper portion 126 other than the separation channel or channels.

In illustrative embodiments of systems discussed herein, since the quality of semen samples varies, the number of motile sperm harvested or collected from each of the collection chambers, also typically varies. In some circumstances, in some illustrative embodiments, the number of motile sperm in each of the collection chambers can be similar. For example, in some illustrative embodiments, wherein the semen sample is of relatively higher quality, each of the collection chambers can contain at least 100,000, 500,000, 1,000,000, 5,000,000, or 10,000,000 motile sperm, or between 100,000 and 10,000,000, 1,000,000,000 or 500,000 motile sperm. In a contrasting example, in some illustrative embodiments, wherein the semen sample is of relatively lower quality, each of the collection chambers can contain between 100 and 1,000, 5,000, or 10,000 motile sperm. In some illustrative embodiments, each of the collection chambers can contain a different number of motile sperm, for example between 1.5× on the low end and 2×, 5×, 10×, 100×, 1,000×, 10,000×, 100,000×, or 1,000,000× on the high end, different. For example, in some illustrative embodiments, the collection chamber(s) closest to (i.e., proximal to) the filter chamber can contain relatively higher numbers of motile sperm, while the collection chamber(s) farther from (i.e., distal to) the filter chamber can contain relatively lower numbers of motile sperm. For example, in an illustrative embodiment wherein the system includes three serially connected collection chambers (e.g., similar to the embodiment shown in FIG. 5 but having 3-10 collection chambers, as non-limiting examples, in series each separated by a separation channel), at the end of an incubation period, the first (i.e., most proximal) collection chamber contains the greatest number of motile sperm, the second (i.e., the middle) collection chamber contains an intermediate number of motile sperm, and the third (i.e., the distal) collection chamber contains the lowest number of motile sperm. As a non-limiting example, the first collection chamber can contain 1,000,000 to 10,000,000 motile sperm, the second chamber can contain 1,000 to 100,000 motile sperm, and the third chamber can contain 100 to 500 motile sperm. In still another illustrative embodiment, wherein the system includes a first collection chamber connected to the filter chamber, with three additional collection chambers radiating outwardly therefrom, in a flower arrangement, similar to the embodiment shown in FIG. 6, the first collection chamber can contain the greatest number of motile sperm and all of the additional collection chambers can contain a lower but relatively similar (e.g. +/−20, 15, 10, 5, or 1%) number of motile sperm between them. For example, the first collection chamber can contain a sufficient number of motile for IVF (e.g., 25,000 to 100,000 motile sperm) while the additional chambers can each contain a lower number of motile sperm that is useful for selecting individual sperms for ICSI (e.g., 100 to 1,000 motile sperm+/−10% between some, most, or all of the additional collection chambers).

### Filter

Systems and devices herein typically include a filter in a filter chamber, wherein the filter separates a lower portion from an upper portion of the filter chamber. In illustrative embodiments herein (e.g., FIGS. 1A-1B), the fluidic system 110 is configured such that motile sperm in the lower portion 124 pass through the filter 118 against gravity and into the upper portion 126 of the filter chamber 116. In illustrative embodiments, at least some of these sperm then move from the upper portion 126, along the fluidic path, or sperm swim path, and into the collection chamber(s) 125a and/or 125b in illustrative embodiments in a flow-free manner, without the application of an external force. This movement of sperm typically occurs mainly during an incubation period after the sperm have been deposited in the inlet 114 and before sperm is/are withdrawn from an outlet, such as but not limited to one or more of the collection chambers (125a, 125b, 125c, etc.).

In illustrative embodiments, the filter is arranged or disposed between the lower component and upper component. In illustrative embodiments, the filter is arranged horizontally within the filter chamber when a system or device herein containing the filter, is laid flat on a horizontal surface, such as a horizontal table in an ART clinic, such that sperm have to swim up against gravity to swim through the filter. In some embodiments, the filter can extend into the collection chamber (i.e., outlet), and in some embodiments, can be attached to the base of the collection chamber to prevent motile sperm from swimming around the filter instead of through the filter, to get to the upper portion, a separation channel connected to the upper portion, and/or to one or more collection chambers. In some embodiments, the filter can be encased (i.e., framed) in a sub-housing, which can be made of plastic for example and can be attached to the filter by an adhesive material to form a filter subassembly. In some embodiments, the filter can be framed with a DSA border, which can serve to connect the lower component and the upper component. The frame of a filter subassembly can have circular and rectangular opening cut into it to conform to the various channels and chambers that extend through the lower surface of the upper component and/or the top surface of the lower component.

In some embodiments, the filter is located between the upper component (e.g., upper layer) and the lower component (e.g., upper layer). Such embodiments can utilize, for example, a filter subassembly, arranged between, and affixed to, an upper surface of a lower component layer and a lower surface of an upper component layer. In some embodiments, the filter or a subassembly comprising the filter is affixed to either or in illustrative embodiments, both a top surface of the lower component and a bottom surface of the top component. In some embodiments a ledge can be present around the perimeter of a filter chamber in the lower component to form a top surface of the lower component to which the filter or the filter subassembly can be affixed by an adhesive or chemical bond. In such embodiments, the plane of the filter is below the plane of the highest top surface of the lower component. Any of such embodiments can include pillars extending from the bottom of the lower portion of the filter chamber to help support the filter, as disclosed herein.

The filter can be made of any one of a variety of materials, such as polycarbonate (PCTE), polyesthersulfone (PES), polyester (PE), polyester track etched (PETE), PTFE, PEEK, cellulose filter paper, nitrocellulose mixed esters (NCE), nylon, glass fiber, or stainless steel or aluminum filters. In illustrative embodiments, the filter is made of polycarbonate. Membrane pores can be made using techniques known in the art, such as track etching technique, laser drilling, for example. In some embodiments, a mesh or network of pillars can create a tortuous path which can act as a filter.

The system 110 in illustrative embodiments is not connected to an external pumping device that could cause media to flow through the fluidic path to push sperm therethrough unintentionally through the filter for example. Thus, sperm that are input, injected, or delivered into a system or device herein before an incubation period, in illustrative embodiments are separated based upon their swimming ability, so that healthy, more motile sperm are separated from dead sperm, debris, and less motile sperm. Upon harvesting from an outlet, a syringe or pipet can be used to withdraw sperm from the outlet. Although the syringe or pipet may impart some force to the system when it is used to deliver sperm into the inlet before an incubation, or when it is used to extract sperm from the system after the incubation, at that point, at least some of the sperm that are delivered to the inlet will have already traveled through the filter, reached the outlet of interest by swimming, without application of an external force (i.e., in a flow-free manner).

The filter 118 includes a plurality of micropores. The plurality of micropores are sized to permit a head of the sperm to pass therethrough. Mammalian sperm are similar morphologically, with a flat, disk-shaped head, a midpiece, and an elongate tail. However, the sperm of different mammals differ in dimensions. Accordingly, the micropores can be selected for a particular application, such as for human, bovine, equine or other mammalian sperm separation. For example, human sperm has a head that is 5.1 μm by 3.1 μm and a tail that is 50 μm long. Bovine sperm have a head of about 9.2 μm by 5.1 μm and a tail of about 47.5 μm. Equine sperm have a head about 6.0 μm by 3.1 μm and a tail about 49 μm long. Accordingly, the filter micropores, for example depending upon the species of sperm to be introduced/input/applied/delivered into the system 110, can be a target diameter that in some illustrative embodiments, is between 1 μm, 2 μm, 3 μm, 4 μm, 5 μm, 6 μm, 7 μm, or 8 μm on the low end of the range and 25 μm on the high end of the range. In a further example, the plurality of micropores are, or are at least 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, 10.5 μm, 11 μm. 11.5 μm, 12 μm, 12.5 μm, 13 μm, 13.5 μm, 14 μm, 14.5 μm, 15 μm, 15.5 μm, 16 μm, 16.5 μm, 17 μm, 17.5 μm, 18 μm, 18.5 μm, 19 μm, 19.5 μm, 20 μm, 20.5 μm, 21 μm, 21.5 μm, 22 μm, 22.5 μm, 23 μm, 23.5 μm, 24 μm, 24.5 μm, or 25 μm in diameter. In some embodiments, the plurality of micropores have a diameter greater than 25 μm. In illustrative embodiments, the plurality of micropores have about the same diameter. In some embodiments the plurality of micropores can be a target diameter from 3 μm, 4 μm, 5 μm, 6 μm, 7 μm, or 8 μm on the low end of the range, to 20 μm on the high end of the range. In some embodiments the plurality of micropores can be a target diameter from 3 μm, 4 μm, 5 μm, 6 μm, 7 μm, or 8 μm on the low end of the range, to 18 μm on the high end of the range. In some embodiments the plurality of micropores can be a target diameter from 3 μm, 4 μm, 5 μm, 6 μm, 7 μm, or 8 μm on the low end of the range, to 15 μm on the high end of the range. In some embodiments, the micropores range from about 5 μm to about 10 μm, or from about 8 μm to about 14 μm. In some embodiments, the plurality of micropores include diameters ranges from about 6 μm to about 10 μm, about 3 μm to about 20 μm, about 4 μm to about 20 μm, about 6 μm to about 20 μm, about 7 μm to about 20 μm, about 8 μm to about 20 μm, or about 9 μm to about 20 μm. Typically, the micropores of a filter are of the same target diameter within the manufacturing error range from the target diameter of current filters. For example, the filter diameters can be within 1, 2, 3, 4, 5, 10, 20, or 25% of the target value, for example, wherein the target value is within a target range. In some embodiments, two or more filters 118 are layered to provide a range of micropore diameters, such that at least one layer and in some embodiments all layers have a different diameter micropores than other layers.

In some illustrative embodiments, the plurality of micropores are sized to permit a head of the sperm to pass therethrough. In some illustrative embodiments, the plurality of micropores are between 1 μm and 20 μm in diameter. In some illustrative embodiments, the plurality of micropores are between 6 μm and 18 μm in diameter. In some illustrative embodiments, the filter chamber 116 comprises at least two filters. In some illustrative embodiments, the at least two filters comprise a plurality of micropores of between 1 μm and 20 μm in diameter. In some illustrative embodiments, the at least two filters comprise a plurality of micropores of between 6 μm and 18 μm in diameter. In some illustrative embodiments, the at least two filters comprise a first filter with a plurality of micropores having a first diameter, and a second filter with a plurality of micropores having a second diameter different than the first diameter. In some illustrative embodiments, at least one of the first collection chamber 125a and the second collection chamber (125b) comprises a filter having a plurality of micropores sized to permit a head of the sperm to pass therethrough.

In illustrative embodiments, the filter 118 can be fabricated/manufactured of a wide variety of materials having suitable material properties, as long as the pore sizes can be controlled and/or sized for sorting sperm. Suitable materials include but are not limited to polycarbonate, polyester and nylon. In certain illustrative embodiments, materials other than polycarbonate, polyester and nylon can be used, so long as they have suitable material properties and pore size for sorting sperm, such as described herein.

### Sperm Chemoattractants

It has been demonstrated that in couples where 65% of sperm bound hyaluronic acid, the selection of hyaluronic acid-bound sperm for ICSI led to a statistically significant reduction in pregnancy loss rates (Worrilow, et al., Hum Reprod. 2013 February; 28(2): 306-314). In some embodiments of the system 110, at least one of the first collection chamber 125a and the second collection chamber 125b includes a sperm chemoattractant, such as but not limited to hyaluronic acid, progesterone, chemokine CCL20, atrial natriuretic peptide (ANP), an odorant, natriuretic peptide type C (NPPC), and/or allurin. For example, a surface of one or both of the collection chambers 125a and 125b can be coated with the sperm chemoattractant. In another example, at least one surface of one or both of the collection chambers 125a and 125b includes a microdot containing the sperm chemoattractant. Typically, the sperm chemoattractant is rehydrated by the addition of fluid, such as a buffer. In some illustrative embodiments, the sperm chemoattractant is applied to the surface(s) so as to create a gradient of increasing concentration, such that the concentration of the sperm chemoattractant increases as the sperm swim toward the collection chamber. In some illustrative embodiments, the gradient of sperm chemoattractant is a dynamic gradient over the length of the channel, system or device.

In some illustrative embodiments, a surface of one or the chambers and/or one of the channels comprises a sperm chemoattractant. In some illustrative embodiments, a surface of one or more of the collection chambers in the post filter zone is coated with a sperm chemoattractant. In some illustrative embodiments, a surface of one or more of the channels in the post filter zone is coated with a sperm chemoattractant. In some illustrative embodiments, the sperm chemoattractant is present on the surface in a concentration gradient. In some illustrative embodiments, the sperm chemoattractant is at least one of hyaluronic acid, progesterone, chemokine CCL20, atrial natriuretic peptide (ANP), an odorant, natriuretic peptide type C (NPPC), and allurin. In other embodiments, the sperm chemoattractant is at least one of the sperm chemoattractant is at least one of hyaluronic acid, progesterone, chemokine CCL20, atrial natriuretic peptide (ANP), natriuretic peptide type C (NPPC), and allurin. In some illustrative embodiments, the sperm chemoattractant is within a hydrogel that is associated and/or coated with the surface of one or more of the chambers and/or the channels. For example, in some illustrative embodiments, the sperm chemoattractant is suspended, dissolved, and/or dispersed in a hydrogel that is applied to one or more surfaces of the one or the chambers and/or the one of the channels. In some illustrative embodiments, the sperm chemoattractant is released as the sperm traverse or progress along the fluidic path through the fluidic system. For example, in an illustrative embodiment, the sperm chemoattractant dissolves into a liquid media filling the separation channel(s) and/or the collection chamber(s) into the fluid path, such as but not limited to so as to attract and/or draw the sperm toward the collection chamber(s). For example, in an illustrative embodiment, the sperm chemoattractant is applied to a collection chamber, such as in a hydrogel formulation, which then dries after manufacture of the system. Prior to pipetting a sample of sperm into the inlet, the system is filled with a buffered liquid media, such as for example for the sperm to swim through. The buffered liquid media hydrates the dried hydrogel, thereby enabling and/or allowing at least some of the sperm chemoattractant to diffuse into the adjacent buffered liquid media. In some illustrative embodiments, the sperm chemoattractant can diffuse into the buffered liquid media over a period of time, and thereby create a concentration gradient of the sperm chemoattractant within the one or more of the chambers and/or the channels. In some embodiments, the concentration of the gradient of the sperm chemoattractant is, in some illustrative embodiments, the gradient of sperm chemoattractant is a dynamic gradient over the length of the channel, system or device, and/or a dynamic gradient over time. In some embodiments the concentration of the sperm chemoattractant gradient increases over time and/or the length of the channel, system or device. In some embodiments, the concentration gradient of the sperm chemoattractant decreases over time and/or the length of the channel, system or device. In some embodiments, the gradient of the chemoattractant is not dynamic, but is fixed, stable, or maintained over the length of the channel, system or device, and/or does not change over time. In some embodiments, the concentration gradient of the chemoattractant is fixed, stable, or maintained over time and/or the length of the channel, system or device.

In some illustrative embodiments, a surface of one or both of the first collection chamber 125a and the second collection chamber 125b comprises a sperm chemoattractant so as to provide one or more sperm chemoattractant-coated surfaces, and wherein the method further comprises before the harvesting, allowing at least one of the motile sperm in one or both the first collection chamber 125a and the second collection chamber 125b to bind the one or more sperm chemoattractant-coated surfaces.

In some illustrative embodiments, the harvesting of the sperm is performed by collecting at least one of the motile sperm bound to the sperm chemoattractant-coated surfaces. For example, in some illustrative embodiments, some, most, virtually all or all of the sperm bound to the sperm chemoattractant-coated surfaces are collected, withdrawn and/or harvested.

### Imaging Sub-Assemblies

In some embodiments, systems herein further comprise an imaging device, imaging assembly, imaging subassembly, imaging means, or imaging function. Such imaging device when present is typically optically connected to at least one channel or chamber of a sperm sorting system herein, and in illustrative embodiments is at least optically connected to 1, 2 or more, or a plurality of collection chambers. Using such an imaging device, motile sperm can be detected, for example in 1, 2, or more collection chambers, optically, e.g. either visually or using an optical device, such as a microscope, through the top or bottom (e.g. through a transparent region of the substrate) of a system herein. As disclosed herein, the top surface of a filter chamber or a collection chamber herein can be open, or if overlaid with a cover, such cover can be transparent. Furthermore, the support of a sperm-sorting system or device herein can be transparent. Thus, an individual, technician, or practitioner can view sperm in the system or device with a microscope, such as a conventional microscope. Furthermore, optical probing can be performed either visually or by analyzing optical signals, such as images, generated from within the sperm-sorting system or device. Such visual or optical signal analysis typically involves viewing and/or counting a sperm or subpopulation of sperm as they move through a system therein and/or as they are located in 1, 2, or more collection chambers.

### Methods of Sorting Sperm Using a Multi-Well System

The systems of FIG. 1A through FIG. 7, or similar systems that share one or more components of such systems, can be used to facilitate the movement of sperm, and to direct healthy sperm to a collection chamber in illustrative embodiments in a flow free manner, and typically are used to sort sperm, wherein the sperm are typically sorted based upon their swimming ability, such that more motile sperm, or healthier and in illustrative embodiments some, most, virtually all or all of the most motile and presumably healthiest sperm can be detected, counted, and in illustrative embodiments, harvested. For example, a sperm sample, which in illustrative embodiments is semen, typically a liquified semen sample, can be introduced, inserted, injected or otherwise delivered into an inlet of a system or device that includes a filter chamber separating a pre-filter zone and a post-filter zone that includes a separation channel typically connecting a collection chamber to the filter chamber. In illustrative embodiments such post-filter zone includes two or more (e.g. a plurality of) separation channels and collection chambers. In illustrative embodiments, each separation channel is connected to a different collection chamber. Thus, in such a method, sperm that swim through the system, in illustrative embodiments in a flow-free manner to a collection chamber can be detected, counted, and/or harvested with a syringe or a pipet (e.g., volumetric pipet or Pasteur pipet, etc.). In illustrative embodiments, sperm are harvested with a syringe. Although withdrawal of liquid from a collection chamber of the system or device using the syringe or pipet imparts some force on liquid within the system or device, in illustrative embodiments at least some of the sperm will have already traveled through the filter and reached the outlet or collection chamber of interest by their own motile ability by swimming to the collection chamber of interest in a flow-free manner (i.e., without application of an external force). For example, between 5% and 10, 15, 20, 25, 30, 40, 50, 60, 70, 75, 80, 85, 90, 95%, 99%, or 100% of the motile sperm in the sample that is delivered to the outlet swim to the outlet (e.g., target collection chamber) during an incubation period, before sperm at the target collection chamber are harvested.

In illustrative embodiments, an individual, technician or practitioner can choose the collection chamber from which they harvest sperm using any of the robust and flexible systems provided herein depending on the number of motile sperm in each of the two or more collection chambers and optionally an ART procedure that is being contemplated for harvested sperm.

Accordingly, in an illustrative embodiment, a method for sorting sperm is provided, including the steps of delivering a sample of sperm 01 into an inlet (e.g., 114, 214, 314, 414, 514, 614, 714) connected to a fluidic system (e.g., 110, 210, 310, 410, 510, 610, 710), such as is shown in FIGS. 1(A and B) to 7, and then allowing the sperm in the sample of sperm to traverse a fluidic path through the fluidic system without application of an external force (i.e., in a flow-free manner) (e.g., 110, 210, 310, 410, 510, 610, 710) from the inlet (e.g., 114, 214, 314, 414, 514, 614, 714) through at least one introduction channel (e.g., 120, 220, 320, 420, 520, 620, 720) into a filter chamber (e.g., 116, 216, 316, 416, 516, 616, 716). The filter chamber (e.g., 116, 216, 316, 416, 516, 616, 716) includes a lower portion (e.g., 124, 224, 324, 424, 524, 624, 724) and an upper portion (e.g., 126, 226, 326, 426, 526, 626, 726) positioned above the lower portion (e.g., 124, 224, 324, 424, 524, 624, 724), and the lower portion (e.g., 124, 224, 324, 424, 524, 624, 724) and the upper portion (e.g., 126, 226, 326, 426, 526, 626, 726) are separated by a filter (e.g., 118, 218, 318, 418, 518, 618, 718) positioned in the filter chamber (e.g., 116, 216, 316, 416, 516, 616, 716). The filter (e.g., 118, 218, 318, 418, 518, 618, 718) has a plurality of micropores sized to permit a head of the sperm to pass therethrough, such as is discussed herein. Then, in illustrative embodiments without the application of an external force, at least some motile sperm from the sample of sperm that have entered the lower portion (e.g., 124, 224, 324, 424, 524, 624, 724) of the filter chamber (e.g., 116, 216, 316, 416, 516, 616, 716) are allowed to selectively pass through the filter (e.g., 118, 218, 318, 418, 518, 618, 718) typically against gravity, so as to traverse the fluidic path into the upper portion (e.g., 126, 226, 326, 426, 526, 626, 726) of the filter chamber (e.g., 116, 216, 316, 416, 516, 616, 716), and then to traverse the fluidic path through a first separation channel (e.g., 127a, 227a, 327a, 427a, 527a, 627a, 727a) into a first collection chamber (e.g., 125a, 225a, 325a, 425a, 525a, 625a, 725a) typically such that a percentage of motile sperm present within the first collection chamber (e.g., 125a, 225a, 325a, 425a, 525a, 625a, 725a) is greater than a percentage of motile sperm within the sample. In illustrative embodiments, this is then followed by either (a) allowing at least one of the motile sperm in the first collection chamber (e.g., 125a, 225a, 325a, 425a, 525a, 625a, 725a) to further traverse through a second separation channel in a flow-free manner (e.g., 127b, 227b, 327b, 427b, 527b, 627b, 727b) into a second collection chamber (e.g., 125b, 225b, 325b, 425b, 525b, 625b, 725b), in further illustrative embodiments without the application of an external force; or (b) allowing at least one of the motile sperm from the upper portion 126 to traverse through a second separation channel in a flow-free manner (e.g., 127b, 227b, 327b, 427b, 527b, 627b, 727b) connected directly to the upper portion (e.g., 126, 226, 326, 426, 526, 626, 726) of the filter chamber (e.g., 116, 216, 316, 416, 516, 616, 716), into a second collection chamber (e.g., 125b, 225b, 325b, 425b, 525b, 625b, 725b, in further illustrative embodiments without the application of an external force, wherein the second separation channel (e.g., 127b, 227b, 327b, 427b, 527b, 627b, 727b) is longer than, for example at least 1.1, 125, 1.5, 1.75, 2, 2.5, 3, 4, 5, 10× the length of, the first separation channel (e.g., 127a, 227a, 327a, 427a, 527a, 627a, 727a). Next, the method typically includes the step of harvesting at least one and typically some of the motile sperm that have passed into one or both the first collection chamber (e.g., 125a, 225a, 325a, 425a, 525a, 625a, 725a) and the second collection chamber (e.g., 125b, 225b, 325b, 425b, 525b, 625b, 725b. In certain embodiments, the first and second separation channel and collection chamber are members of a plurality, set, or collection of separation channels and collection chambers, wherein in illustrative embodiments at least one of such separation channels is longer than at least one other separation channel, and wherein at least one, and in some embodiments two or more and in certain embodiments all of the separation channels are connected to the filter chamber and the other separation channels are connected to collection chambers on both ends.

In another illustrative embodiment, a method for sorting sperm is provided, including delivering a sample of sperm to an inlet (e.g., 114, 214, 314, 414, 514, 614, 714) connected to a fluidic system (e.g., 110, 210, 310, 410, 510, 610, 710). The fluidic system comprises the inlet (e.g., 114, 214, 314, 414, 514, 614, 714), a pre-filter zone (e.g., 130, 230, 330, 430, 530, 630, 730) and a post-filter zone (e.g., 128, 228, 328, 428, 528, 628, 728). The pre-filter zone comprises at least one introduction channel (e.g., 120, 220, 320, 420, 520, 620, 720) that extends from the inlet (e.g., 114, 214, 314, 414, 514, 614, 714) to a filter chamber (e.g., 116, 216, 316, 416, 516, 616, 716), a filter (e.g., 118, 218, 318, 418, 518, 618, 718) having a plurality of micropores and arranged in the filter chamber (e.g., 116, 216, 316, 416, 516, 616, 716). The post filter zone 128 has a first post-filter separation channel (e.g., 127a, 227a, 327a, 427a, 527a, 627a, 727a) connected to a first collection chamber (e.g., 125a, 225a, 325a, 425a, 525a, 625a, 725a) configured to facilitate harvesting some of the motile sperm therein. Either or both (a) the pre-filter zone is configured and/or adapted to sort sperm based on their swimming ability and is other than a single straight channel, and (b) the post filter zone (e.g., 128, 228, 328, 428, 528, 628, 728) is configured and/or adapted to sort sperm based on their swimming ability and is other than a single straight separation channel connected to a single collection chamber. Then, without the application of external force, sperm delivered to the fluidic system through the inlet (e.g., 114, 214, 314, 414, 514, 614, 714) are allowed to progress along a fluidic path toward the filter chamber (e.g., 116, 216, 316, 416, 516, 616, 716), and then to move through the filter (e.g., 118, 218, 318, 418, 518, 618, 718) and against gravity to the post-filter zone (e.g., 128, 228, 328, 428, 528, 628, 728), and then to move through the post-filter zone (e.g., 128, 228, 328, 428, 528, 628, 728) to the first collection chamber (e.g., 125a, 225a, 325a, 425a, 525a, 625a, 725a). At least some motile sperm are harvested from the first collection chamber (e.g., 125a, 225a, 325a, 425a, 525a, 625a, 725a).

In another illustrative embodiment, motile sperm delivered to the inlet (e.g., 114, 214, 314, 414, 514, 614, 714) travel a first distance through the first separation channel (e.g., 127a, 227a, 327a, 427a, 527a, 627a, 727a) and a second distance through the second separation channel (e.g., 127b, 227b, 327b, 427b, 527b, 627b, 727b), wherein the second distance is at least 1.5× greater than the first distance.

In certain circumstances, it is desirable to sort the sperm more than once. In some illustrative embodiments, the method of sorting sperm is performed a second time with a second fluidic system (e.g., 110, 210, 310, 410, 510, 610, 710) that is identical to the first fluidic system (e.g., 110, 210, 310, 410, 510, 610, 710). In some illustrative embodiments, when performing the method the first time, motile sperm are collected from the first collection chamber (e.g., 125a, 225a, 325a, 425a, 525a, 625a, 725a) of the first fluidic system (e.g., 110, 210, 310, 410, 510, 610, 710) and then, when performing the method the second time, motile sperm are collected from the second collection chamber 125b of the second fluidic system. In a further illustrative embodiment, at least 1000× or more motile sperm are collected from the first collection chamber (e.g., 125a, 225a, 325a, 425a, 525a, 625a, 725a) of the first fluidic system, than are collected from the second collection chamber (e.g., 125b, 225b, 325b, 425b, 525b, 625b, 725b) of the second fluidic system. In a further illustrative embodiment, a range of motile sperm are collected from the first collection chamber, such as at least 1,000× more motile sperm at the low end of the range to at least 2,000×, 3,000×, 5,000×, or 10,000× more motile sperm at the high end of the range, than are collected from the second collection chamber. In some illustrative embodiments, at least 100,000× more motile sperm are collected from the first collection chamber, than are collected from the second collection chamber.

In some illustrative embodiments, when the at least some of the motile sperm are harvested, a higher percentage of motile sperm are present within the first collection chamber (e.g., 125a, 225a, 325a, 425a, 525a, 625a, 725a) than in the sample. In some illustrative embodiments, when the at least some of the motile sperm are harvested, the first collection chamber (e.g., 125a, 225a, 325a, 425a, 525a, 625a, 725a) and the second collection chamber (e.g., 125b, 225b, 325b, 425b, 525b, 625b, 725b) do not comprise a filter.

In some illustrative embodiments, atop portion of the upper portion (e.g., 126, 226, 326, 426, 526, 626, 726) of the filter chamber (e.g., 116, 216, 316, 416, 516, 616, 716), a top portion of the first collection chamber (e.g., 125a, 225a, 325a, 425a, 525a, 625a, 725a) and atop portion of the second collection chamber (e.g., 125b, 225b, 325b, 425b, 525b, 625b, 725b) are open outlets that provide access to the fluidic system.

In some illustrative embodiments, at least 1000× more motile sperm are collected from the first collection chamber (e.g., 125a, 225a, 325a, 425a, 525a, 625a, 725a) than the second collection chamber 125b.

In some illustrative embodiments, the first collection chamber (e.g., 125a, 225a, 325a, 425a, 525a, 625a, 725a) is connected to the second collection chamber (e.g., 125b, 225b, 325b, 425b, 525b, 625b, 725b) by the second separation channel (e.g., 127b, 227b, 327b, 427b, 527b, 627b, 727b).

In some illustrative embodiments, the at least one of the motile sperm from the upper portion (e.g., 126, 226, 326, 426, 526, 626, 726) traverse through the second separation channel (e.g., 127b, 227b, 327b, 427b, 527b, 627b, 727b) connected directly to the upper portion (e.g., 126, 226, 326, 426, 526, 626, 726), into the second collection chamber (e.g., 125b, 225b, 325b, 425b, 525b, 625b, 725b) in illustrative embodiments without the application of an external force, wherein the second separation channel (e.g., 127b, 227b, 327b, 427b, 527b, 627b, 727b) is between 1.25× or 1.5× to 10× the length of the first fluidic connection channel (e.g., 127a, 227a, 327a, 427a, 527a, 627a, 727a).

In some illustrative embodiments, the method is performed at least two times, a first time using the first fluidic system (e.g., 110, 210, 310, 410, 510, 610, 710) and a second time using a second fluidic system (e.g., 110, 210, 310, 410, 510, 610, 710), wherein the first fluidic system (e.g., 110, 210, 310, 410, 510, 610, 710) and the second fluidic system (e.g., 110, 210, 310, 410, 510, 610, 710) are identical in design, configuration and assembly, and wherein one of the times between 1 million and 25 million sperm are collected from the first collection chamber (e.g., 125a, 225a, 325a, 425a, 525a, 625a, 725a) and the other time less than 1 million, less than 100,000, less than 10,000, less than 1,000, less than 100, or less than 10 sperm are collected from the second collection chamber (e.g., 125b, 225b, 325b, 425b, 525b, 625b, 725b).

In some illustrative embodiments, the method is used to collect 100,000, or 1, 2, 3, 4, 5, 6, 7, 8, 9, or 10 million or more sperm, for example for an artificial insemination procedure and in another performance of the same method using a system or device that is identical in configuration, design and assembly, less than 1,000,000, 100,000, 10,000, 1,000, 100, or 10 sperm are collected, for example for use in an in vitro fertilization procedure, for example, less than 10,000, 1,000, 100, or 10 sperm for use in an ICSI procedure.

In some illustrative embodiments, the volume of a sample of sperm delivered to a system or device in a method herein is greater than 10, 20, 30, 40, or 50 μl. For example, the sample can be 50 μl on the lower end to about 100 μl, 250 μl, 500 μl, 1 ml, 2 ml, 3 ml, 4 ml, 5 ml 6 ml, 7 ml, 8 ml, 9 ml, 10 ml, 15 ml, 20 ml or 25 ml on the high end.

Methods and systems provided herein, in illustrative embodiments, sort sperm rapidly, such as in less than 60, 45, or 30 minutes, for example between 15 minutes and 60, 45, or 30 minutes, without the need for complex instrumentation or trained operators. In some embodiments, the period of time between when sperm are delivered to an inlet of a system herein and harvested from the system, for example from a collection chamber, is a period of time ranging from about 5 minutes at the lower end, to 24, 12, 8, 4, 2, or 1 hour, or 45, 30, 15, or 10 minutes on the high end of the range. In some embodiments, such period of time ranges from about 15 minutes at the lower end, to 24, 12, 8, 4, 2, or 1 hour, or 45, or 30 minutes on the high end of the range. In certain illustrative embodiments, such period of time ranges from 20 minutes on the low end, and 45, 40, 35 or 30 minutes on the high end, or between 25 minutes on the low end and 45, 40, 35, or 30 minutes on the high end.

In some embodiments, an individual, technician, practitioner or physician performing a method for sorting sperm herein observes sperm as they travel through the system or device and/or as they move to one or more collection chambers, for example using an imaging subassembly, function, and/or means optically or visually connected to a sperm sorting system or device provided herein. Such individual, technician, practitioner or physician then typically harvests sorted sperm when 1 or a group of sorted sperm reach a target collection chamber, or a desired number, or an approximate desired number, of sperm reach a collection chamber or a target collection chamber depending on a particular assisted reproductive technology (ART) procedure to be performed using the harvested sperm. Such particular ART method can be predetermined, or it can be determined based on the number of sperm harvested from a collection chamber(s) such as a target collection chamber, or that are observed within a collection chamber by such individual, technician, practitioner or physician performing the method. In some embodiments, a method herein can be performed without a predetermined target sperm number, without a target collection chamber, and without a preselected ART procedure.

In some illustrative embodiments, the method further includes performing an ART procedure using at least one and typically a population of the harvested sperm. For example, regardless of whether the initial sperm sample has millions or 10s of millions of healthy, motile sperm or just a few healthy, motile sperm, the practitioner is able to sort the sperm sample, and then perform an ART procedure that can be selected, for example based on the numbers of sperm that are harvested in one, two or more collection chambers. Healthy sperm counts from a sorted healthy sample can vary greatly depending on the quality of the initial sperm sample. Depending on the configuration of separation channels and collection chambers in a sperm separation system used to perform the method, as well as the number of motile sperm in a sample, the number of collection chambers with motile sperm for harvesting can range from 1 to 12 or more, and in illustrative embodiments is 2, at least 2, or between 2 on the low end and 6, 5, 4, or 3 on the high end.

The ART procedure performed in embodiments herein that include a step of performing an ART procedure, include any ART procedure known in the art. For example, the ART procedure can be artificial insemination, which in illustrative embodiments is intrauterine insemination (IUI), or the ART procedure can be in vitro fertilization (IVF), or intracytoplasmic sperm injection (ICSI). Thus, in some embodiments, at least 100,000, 250,000, 500,000, 1,000,000, 2,500,000, 5,000,000, 7,500,000, or 10,000,000 sperm are harvested and the ART procedure is IUI. In some embodiments, less than 1,000,000, 750,000, 500,000, 250,000, 100,000, 50,000, 25,000 or 10,000 sperm are harvested and the ART procedure is in vitro fertilization. In some embodiments, less than 100,000, 50,000, 25,000, 10,000, 5,000, 2,500, 1,000, 500, 250, 100, 50, 25, or 10 sperm are harvested and the ART procedure is ICSI.

In some embodiments, as a non-limiting example using the system of FIG. 6 or a similar system with 2 or more collection chambers that are each connected to the same chamber (e.g. filter chamber or another collection chamber) by separation channels having dimensions that are within 20, 15, 10, 5, 1, or identical to each other, at least 1,000, 10,000, 50,000, 100,000, 500,000, 1,000,000, 5,000,000, or 10,000,000 motile sperm are harvested from each of at least 2, 3, 4, 5, 6, 7, 8, 9, 10, 11 or 12 collection chambers. In some embodiments, as non-limiting examples see the embodiments of FIGS. 1-5 and 7 or a similar system having 2 or more collection chambers that are each connected in a series to each other through a separation channel, or that are connected to the same chamber but have different (e.g. at least 25, 30, 40, 50, 75% different, lengths, widths or diameters or 1.25, 1.5, 2, 3, 4, or 5× different lengths), at least 100,000, 500,000, 1,000,000, 5,000,000, or 10,000,000 motile sperm are harvested from a collection chamber that is proximal to the filter chamber (or another collection chamber) compared to a collection chamber from which less than 100,000 motile sperm are harvested. Motile sperm harvested from more than 1 collection chamber can be combined and a portion or all of the combined, harvested motile sperm sample can be used in an ART procedure. Harvested motile sperm samples from 1 or more of the collection chambers either combined or individually can be frozen in cryovials and cryopreservation medium for later use.

In illustrative embodiments of systems discussed herein, since the quality of semen samples varies, the number of motile sperm harvested or collected from each of the collection chambers, also typically varies. In some circumstances, in some illustrative embodiments, the number of motile sperm harvested from each of the collection chambers can be similar. For example, in some illustrative embodiments, wherein the semen sample is of relatively higher quality, at least 100,000, 500,000, 1,000,000, 5,000,000, or 10,000,000 motile sperm, or between 100,000 and 10,000,000, 1,000,000,000 or 500,000 motile sperm can be harvested from each of the collection chambers. In a contrasting example, in some illustrative embodiments, wherein the semen sample is of relatively lower quality, between 100 and 1,000, 5,000, or 10,000 motile sperm can be harvested from each of the collection chambers. In some illustrative embodiments, the number of motile sperm harvested from each of the collection chambers can be different, for example between 1.5× on the low end and 2×, 5×, 10×, 100×, 1,000×, 10,000×, 100,000×, or 1,000,000× on the high end, different. For example, in some illustrative embodiments, relatively higher numbers of motile sperm can be harvested from the collection chamber(s) closest to (i.e., proximal to) the filter chamber, while relatively lower numbers of motile sperm can be harvested from the collection chamber(s) farther from (i.e., distal to) the filter chamber. For example, in an illustrative embodiment wherein the system includes three serially connected collection chambers (e.g., similar to the embodiment shown in FIG. 5 but having 3-10 collection chambers, as non-limiting examples, in series each separated by a separation channel), at the end of an incubation period, the greatest number of motile sperm can be harvested from the first (i.e., most proximal) collection chamber, an intermediate number of motile sperm can be harvested from the second (i.e., the middle) collection chamber, and the lowest number of motile sperm can be harvested from the third (i.e., the distal) collection chamber. As a non-limiting example, 1,000,000 to 10,000,000 motile sperm can be harvested from the first collection chamber 1,000 to 100,000 motile sperm can be harvested from the second chamber, and 100 to 500 motile sperm can be harvested from the third chamber. In still another illustrative embodiment, wherein the system includes a first collection chamber connected to the filter chamber, with three additional collection chambers radiating outwardly therefrom, in a flower arrangement, similar to the embodiment shown in FIG. 6, the greatest number of motile sperm can be harvested from the first collection chamber and a lower but relatively similar (e.g. +/−20, 15, 10, 5, or 1%) number of motile sperm can be harvested from each of the additional collection chambers. For example, a sufficient number of motile for IVF (e.g., 25,000 to 100,000 motile sperm) can be harvested from the first collection chamber while a lower number of motile sperm that is useful for selecting individual sperms for ICSI (e.g., 100 to 1,000 motile sperm+/−10% between some, most, or all of the additional collection chambers) can be harvested from each of the additional chambers.

In certain illustrated embodiments, while performing the method, 1,000,000 or more sperm are harvested from one collection chamber, and also 1-99 or fewer sperm are harvested from a different collection chamber (e.g., during the same performance of the method). It is foreseen that these numbers can vary, such as discussed herein. In another illustrated embodiment, during the same performance of the method, at least 100,000 sperm are harvested from at least two collection chambers, such as but not limited to two of the two or more collection chambers.

Further, since in such embodiments there are multiple isolated sperm samples, repeated attempts using the same ART procedure, or more than one ART procedure can be performed using the sperm harvested from one collection chamber for a first ART procedure and using the sperm harvested from another collection chamber for a repeat of that same ART procedure, or for performing a different ART procedure. For example, the disclosed methods can be used to isolate sperm from multiple sorted samples from the same initial sample in the same sorting run or sorting method performance; with similar (e.g. +/−20, 15, 10, or 5%) healthy sperm counts in two or more harvested samples from different collection chambers, as a non-limiting example, using the system illustrated in FIG. 6; or with very different (e.g. 1 or more samples having 25%, 50% or 100%, or 1.5, 2, 3, 4, 5, 6, 7, 8, 9, 10, 100, 500, 1,000, 5,000, 10,000, 100,000, 1,000,000, 2,000,000, 3,000,000,000, 4,000,000,000, or 5,000,000 times more) numbers of motile sperm than another harvested sample from different collection chambers, as a non-limiting example using a system such as that shown in FIGS. 1-5.

In an example, the practitioner uses the system 110 shown in FIGS. 1A-1B to sort a patient's sperm sample sorted into two samples of motile, healthy, sorted sperm. For example, the system 110 is loaded with a buffered solution, and then as a non-limiting example 100 μl to 500 μl of the patient's sperm sample is pipetted into the inlet 114 in the pre-filter zone 130. The system 110 is then incubated for a period of time, such as 30-40 minutes, during which time the sperm 01 self-sort themselves based upon their respective swimming abilities. Namely, the sperm 01 swim from the inlet 114, through the introduction channel 120 in the pre-filter zone 130 and into the filter chamber 116, in illustrative embodiments without the application of an external force. Once in the filter chamber 116, some sperm 02 are able to swim upward through the pores in the filter 118 against gravity so as to traverse the fluidic path into the upper portion of the filter chamber (e.g., from the lower portion 124 into the upper portion 126). At this point, if the upper portion 126 includes an orifice, the practitioner can elect to harvest some or all of the sorted sperm 02 from the upper portion 126 of the filter chamber 116, for example to use in an in vivo ART procedure, such as IUI. After reaching the upper portion 126, a portion of the sperm 02 therein continue to traverse to the post-filter zone 128 comprising multiple separation channels and collection chambers. The motile sperm self-sort themselves by proceeding into the first separation channel 127a and swimming to the first collection chamber 125a. The number of healthy sperm in the first collection chamber 125a can be from only a few sperm to 10s of millions of sperm, depending upon the quality of the initial sperm sample. If the practitioner is monitoring the sperm as they are sorting, and observes that there are only a few sperm in the first collection chamber 125a, the practitioner can collect individual sperm from the first collection chamber 125a and proceed immediately to an ICSI procedure. However, generally, there will be many more than a few sperm migrating into the first collection chamber 125a. In the case of a better quality initial sperm sample, sperm counts ranging from about 1 million to 10s of millions of motile sperm can migrate/swim into the first collection chamber 125a. Some of these sperm in the first collection chamber 125a will continue on, and swim through the second separation channel 127b and into the second collection chamber 125b. Alternatively, if a system with a different configuration is used, such as systems illustrated in FIG. 2 or FIG. 7, the motile sperm from the upper portion of the filter chamber can transverse through a second separation channel 127b to a second collection chamber 125b directly from the upper portion 126 of the filter chamber 116 in illustrative embodiments without the application of an external force, wherein the second separation channel is at least 1.25, 1.5, 1.75, 2, 3, 4, 5, or 10× the length of the first separation channel. In general, the sorted motile sperm within the second collection chamber 125b are more sorted, or otherwise purified. In some circumstances, the sorted sperm in the second collection chamber 125b can be healthier, the more motile and morphologically normal, as compared to the sorted sperm in the first collection chamber 125a. In some circumstances, the sorted sperm in both of the collection chambers 125a and 125b will be so numerous that the practitioner can choose to use sperm collected from either or both of the collection chambers 125a and 125b for any ART procedure he chooses. In other circumstances, there will be fewer individual sperm cells in the second collection chamber 125b, than in the first collection chamber 125a. However, the practitioner can choose to use the sperm harvested from each or both of the first and second collection chambers 125a and 125b for an ART procedure, based upon the numbers of sperm in either or both of the first and second collection chambers 125a and 125b.

The number of sorted sperm 02 in each of the first and second collection chambers 125a and 125b will vary, depending upon the quality of the initial sperm sample. Thus, at the end of the incubation period, the individual, technician or practitioner can compare the sorted sperm in the collection chambers 125a and 125b, such as by observing them under the microscope or performing various sperm quality analyses known in the art. After incubation, the sorted sperm 02 in one or both of the collection chambers 125a and 125b is collected (i.e., harvested) and can be used either directly in an ART procedure (i.e., IVF or AI) or preserved for later use, such as by freezing.

Based upon the number of motile sperm in each of the first and second collection chambers 125a and 125b, the practitioner can choose to perform an appropriate ART procedure, or to freeze one or both samples for later use. For example, if one or both of the first and second collection chambers 125a and 125b contains 100,000, 500,000, or 1.0 million or more healthy sperm, the practitioner can elect to use the sperm in either or both of the collection chambers 125a and 125b for example for IUI. The sorted sperm from both collection chambers 125a and 125b can be combined/pooled for such a procedure. Alternatively, the sperm from only one of the collection chambers can be used for the IUI and the sperm from the other collection chamber frozen as a backup for a second try at insemination or another ART procedure.

In some illustrative embodiments, the ART procedure is chosen based on the number of sperm harvested from one or more collection chambers 125a and 125b. In a first example, suppose the patient's sperm sample is of sufficient quality and 1-50 million or more sperm are collected from the second collection chamber 125b, then the physician may elect to proceed with an IUI procedure using that sample, while freezing the sample from the first collection chamber 125a as a backup. In a second example, if the sperm sample is of lesser quality, and less than 1 million (e.g. 0.9, 0.8, 0.7, 0.6, 0.5, 0.4, 0.3, 0.2, or 0.1 million) sperm are collected from the second collection chamber 125b, but there are 1.0 million sperm collected in the first collection chamber 125a. Then, the physician can choose between ART procedures. For example, the sperm from the second collection chamber 125b can be used in an in vitro procedure, such as incubating eggs from the mother with the collected sperm in a petri dish, and then implanting one or more of the eggs in the mother. Alternatively, the physician can select several individual sperm from the second collection chamber 125b for ICSI. Further, the physician can elect to use the sperm in the first collection chamber 125a for IUI, and to preserve the sperm in the second collection chamber 125b for a backup in vitro procedure. In a third example, suppose the patient's sperm sample is poor quality, and only 1,000, 500, 100, or 10 motile sperm are collected in the second collection chamber 125b and 0.5 or 0.25 million in the first collection chamber 125a. In this circumstance, the ART procedures are limited to in vitro procedures. Thus, the physician can elect to proceed directly to ICSI, selecting sperm from the second collection chamber 125b. Alternatively, the physician can elect to perform IVF using the motile sperm sample from the first collection chamber 125a. Systems and methods herein provide a physician the ability to select the ART procedure to use, based upon the number of motile sperm in the collection chambers supplies the physician the ability to customize the ART procedure, thereby increasing the likelihood of a successful pregnancy and the best patient outcome possible.

As is discussed above, male fertility is declining, making it more difficult for couples to successfully conceive a healthy and successful pregnancy. However, the fluidic systems (e.g., 110, 210, 310, 410, 510, 610, 710) discussed herein provide the advantage of permitting the selection of a fertilization procedure, depending upon the quality of the sperm sample used, that is more likely to result in a successful pregnancy. For example, if the sperm sample is of higher quality, and the most distal collection chamber (e.g., the second collection chamber, after sorting) contains more than 1 million sperm, and the practitioner can elect to perform intrauterine insemination (IUI), since it has been reported that 1 million or more washed sperm can produce a healthy pregnancy using IUI (Starosta et al., Fertility Research and Practice, 6:23 (2020)). If, after sorting, the proximal collection chamber (e.g., the collection chamber closer to the filter chamber) to the one used for initial harvesting, contains 0.5 million sperm, the practitioner can elect to perform IUI with the sperm harvested from the proximal collection chamber, and possibly freeze and save the sperm harvested from the distal collection chamber for in vitro fertilization procedures, such as for a backup. If the number of sperm in the distal and/or proximal collection chamber is 50-thousand or fewer, the practitioner can elect to perform an in vitro procedure, such as IVF, where the eggs and sperm are incubated together in a container to allow the eggs to become fertilized. If the sperm harvested from the distal and/or proximal collection chamber is 20-thousand or fewer, the practitioner can elect to perform intracytoplasmic injection (ICSI) and examine the harvested 20-thousand sperm for the best 1, 5, 10, 15, 20 or 30 sperm, each of which can be injected directly into an egg to fertilize it.

In some illustrative embodiments, the method further comprises performing an assisted reproductive technology (ART) procedure using at least one of the harvested sperm. In some further embodiments, the ART procedure is chosen based on the number of sperm harvested from one or more collection chambers, such as is discussed above. For example, in some illustrative embodiments, the ART procedure is selected from an intrauterine insemination (IUI), an in vitro fertilization (IVF), and an intracytoplasmic sperm injection (ICSI). For example, in some illustrative embodiments, wherein at least 1,000,000 sperm are harvested, the ART procedure is IUI. In another example, in some illustrative embodiments, wherein less than 1,000,000 sperm are harvested, the ART procedure is in vitro fertilization (i.e., conventional insemination). In yet another example, in some illustrative embodiments, wherein less than 10,000 sperm are harvested and the ART procedure is ICSI. In another example, in some illustrative embodiments, wherein 100,000 or more sperm are harvested from each of at least 2 collection chambers, such as is discussed above. In another example, in some illustrative embodiments, wherein 100,000 or more sperm are harvested from each of at least 3 collection chambers. In another example, in some illustrative embodiments, at least 100,000 sperm are harvested from a collection chamber that is proximal to the filter chamber compared to a collection chamber from which less than 100,000 sperm are harvested. In another example, in some illustrative embodiments, at least 1,000,000 sperm are harvested from a collection chamber that is proximal to the filter chamber compared to a collection chamber from which less than 100,000 sperm are harvested. In another example, in some illustrative embodiments, the post filter zone (128) comprises between 3 and 8 collection chambers. In another example, in some illustrative embodiments, between 3 and 7 of the collection chambers are each connected through their corresponding separation channel, to a first collection chamber that is connected to the filter chamber by a first separation channel.

### Exemplary Embodiments

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects and that can be combined with any other embodiment, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

In one aspect, provided herein is a system for sorting sperm, comprising:
a) a filter chamber comprising a filter comprising a plurality of micropores and arranged between a lower portion and an upper portion positioned above the lower portion; and
b) a post filter zone comprising a first post-filter separation channel connected to a first collection chamber configured to facilitate harvesting some of the motile sperm therein, wherein the post filter zone is other than a single straight separation channel connected to a single collection chamber. In some embodiments, the system further comprises an inlet connected either directly or indirectly to the filter chamber. In illustrative embodiments, the post filter zone has a plurality of separation channels each connected to a different one of a plurality of collection chambers. In some embodiments the filter is arranged horizontally within the filter chamber.

In one aspect, provided herein is a system for sorting sperm, comprising:
a) optionally a housing including a lower component and an upper component coupled together;
b) a fluidic system optionally supported by the housing;
c) an inlet, providing access to the fluidic system to deliver a sample comprising sperm to the fluidic system, and optionally extending through the lower component;
d) a filter chamber configured to pass motile sperm for harvesting and restrict non-motile sperm, the filter chamber comprising a filter comprising a plurality of micropores and arranged between a lower portion and an upper portion positioned above the lower portion;
e) optionally a pre-filter zone comprising at least one introduction channel extending from the inlet to the filter chamber to allow sperm delivered to the fluidic system through the inlet to progress along a fluidic path toward the filter chamber; and
f) a post filter zone comprising a first post-filter separation channel connected to a first collection chamber configured to facilitate harvesting some of the motile sperm therein, wherein either or both
   i) the pre-filter zone is other than a single straight channel; and
   ii) the post filter zone is other than a single straight separation channel connected to a single collection chamber. In some embodiments, the system comprises one or any combination of the optional elements recited in the aspect included in this paragraph, including in illustrative embodiment, all the optional elements. In illustrative embodiments, the post filter zone has a plurality of separation channels each connected to a different one of a plurality of collection chambers.

In another aspect, provided herein is a method for sorting sperm, comprising:
a) delivering a sample of sperm into a filter chamber, wherein the filter chamber includes a lower portion and an upper portion positioned above the lower portion, and wherein the lower portion and the upper portion are separated by a filter positioned in the filter chamber, wherein the filter has micropores sized to permit a head of the sperm to pass therethrough;
c) allowing at least some motile sperm from the sample of sperm that have entered the lower portion of the filter chamber to selectively pass through the filter against gravity in a flow-free manner, so as to traverse the fluidic path into the upper portion of the filter chamber, and then to traverse the fluidic path through a first separation channel into a first collection chamber in a flow-free manner, such that a percentage of motile sperm present within the first collection chamber is greater than a percentage of motile sperm within the sample; either
d1) allowing at least one of the motile sperm in the first collection chamber to further traverse through a second separation channel into a second collection chamber without the application of an external force; or
d2) allowing at least one of the motile sperm from the upper portion to traverse through a second separation channel connected directly to the upper portion of the filter chamber, into a second collection chamber without the application of an external force; and
e) detecting, counting, and/or harvesting at least some of the motile sperm that have passed into one or both the first collection chamber and the second collection chamber. In illustrative embodiments the second separation channel is at least 1.1×, 1.25×, or 1.5× the length of the first separation channel. In some embodiments, the sample is delivered directly into the lower portion of the filter chamber by a user performing the method. For example, the user can insert a pipet or other delivery device within the lower portion to deliver the sample.

In one aspect, provided herein is a system for sorting sperm, comprising:
a) a housing including a lower component and an upper component coupled together;
b) a fluidic system supported by the housing;
c) an inlet extending through the lower component and providing access to the fluidic system to deliver a sample comprising sperm to the fluidic system;
d) a filter chamber configured to pass motile sperm for harvesting and restrict non-motile sperm, the filter chamber including a lower portion extending through the lower component and an upper portion extending through the upper component and positioned above the lower portion;
e) a pre-filter zone comprising at least one introduction channel extending from the inlet to the filter chamber to allow sperm delivered to the fluidic system through the inlet to progress along a fluidic path toward the filter chamber;
f) a filter including a plurality of micropores and arranged in the filter chamber to cause sperm traveling along the fluidic path to move through the filter to reach the upper portion; and
g) a post filter zone comprising a first post-filter separation channel connected to a first collection chamber configured to facilitate harvesting some of the motile sperm therein, wherein either or both
   i) the pre-filter zone is configured and/or adapted to sort sperm based on their swimming ability and is other than a single straight channel; and
   ii) the post filter zone is configured and/or adapted to sort sperm based on their swimming ability and is other than a single straight separation channel connected to a single collection chamber.

In one aspect, provided herein is system for sorting sperm, comprising:
a) a housing including a lower component and an upper component coupled together;
b) a fluidic system supported by the housing;
c) an inlet extending through the lower component and providing access to the fluidic system to deliver a sample comprising sperm to the fluidic system;
d) a filter chamber configured to pass motile sperm for harvesting and restrict non-motile sperm, the filter chamber including a lower portion extending through the lower component and an upper portion extending through the upper component and positioned above the lower portion;
e) at least one introduction channel extending from the inlet to the filter chamber to allow sperm delivered to the fluidic system through the inlet to progress along a fluidic path toward the filter chamber;
f) a filter including a plurality of micropores and arranged in the filter chamber to cause sperm traveling along the fluidic path to move through the filter to reach the upper portion; and
g) a post-filter zone comprising:
   i) a first separation channel connecting the upper portion to a first collection chamber, the first collection chamber being configured to facilitate harvesting some of the motile sperm therein; and
   ii) a second separation channel connected to a second collection chamber, the second collection chamber being configured to facilitate harvesting some of the motile sperm therein, wherein the second separation channel is connected to either the upper portion or the first collection chamber, wherein the first separation channel has a length of less than 5 mm, wherein the second separation channel has a length between 5 and 20 mm, and wherein the length of the second separation channel is at least 1.5 times the length of the first separation channel.

In some embodiments, the second separation channel is connected to the first collection chamber and/or wherein the filter is arranged in the filter chamber to cause sperm traveling along the fluidic path to move through the filter and against gravity to reach the upper portion.

In another aspect, provided herein is a method for sorting sperm, comprising:
a) delivering a sample of sperm into an inlet connected to a fluidic system;
b) allowing sperm in the sample of sperm to traverse a fluidic path through the fluidic system from the inlet through at least one introduction channel into a filter chamber, wherein the filter chamber includes a lower portion and an upper portion positioned above the lower portion, and wherein the lower portion and the upper portion are separated by a filter positioned in the filter chamber, wherein the filter has micropores sized to permit a head of the sperm to pass therethrough;
c) without the application of an external force, allowing at least some motile sperm from the sample of sperm that have entered the lower portion of the filter chamber to selectively pass through the filter against gravity, so as to traverse the fluidic path into the upper portion of the filter chamber, and then to traverse the fluidic path through a first separation channel into a first collection chamber such that a percentage of motile sperm present within the first collection chamber is greater than a percentage of motile sperm within the sample; either
d1) allowing at least one of the motile sperm in the first collection chamber to further traverse through a second separation channel into a second collection chamber without the application of an external force; or
d2) allowing at least one of the motile sperm from the upper portion to traverse through a second separation channel connected directly to the upper portion of the filter chamber, into a second collection chamber without the application of an external force, wherein the second separation channel is at least 1.5× the length of the first separation channel; and
e) harvesting at least some of the motile sperm that have passed into one or both the first collection chamber and the second collection chamber.

In another aspect, provided herein is a method for sorting sperm, comprising:
a) delivering a sample comprising sperm to an inlet connected to a fluidic system comprising the inlet, a pre-filter zone comprising at least one introduction channel extending from the inlet to a filter chamber, a filter having a plurality of micropores and arranged in the filter chamber; and a post filter zone comprising a first post-filter separation channel connected to a first collection chamber configured to facilitate harvesting some of the motile sperm therein, wherein either or both
   i) the pre-filter zone is configured and/or adapted to sort sperm based on their swimming ability and is other than a single straight channel; and
   ii) the post-filter zone is configured and/or adapted to sort sperm based on their swimming ability and is other than a single straight separation channel connected to a single collection chamber;
b) without the application of external force, allowing sperm delivered to the fluidic system through the inlet to progress along a fluidic path toward the filter chamber, and then to move through the filter to the post-filter zone, and then to move through the post-filter zone to the first collection chamber; and
c) harvesting at least some motile sperm from a collection chamber in the post-filter zone.

In some embodiments, the post filter zone comprises a set of separation channels comprising a first separation channel directly connecting to the upper portion and a first collection chamber, a second separation channel, and a third separation channel, wherein each separation channel of the set of separation channels, other than the first separation channel is connected to two collection chambers of a set of collection chambers comprising the first collection chamber, a second collection chamber and a third collection chamber, each collection chamber of the set of collection chambers being configured to facilitate harvesting some of the motile sperm therein, wherein the sum of the lengths of the three or more separation channels is between 15 mm and 100 mm.

In one aspect, provided herein is a system for sorting sperm, comprising:
a) a housing including a lower component and an upper component coupled together;
b) a fluidic system supported by the housing;
c) an inlet. extending through the lower component and providing access to the fluidic system to deliver a sample comprising sperm to the fluidic system;
d) a filter chamber comprising a filter having a plurality of micropores, and arranged between a lower portion and an upper portion positioned above the lower portion;
e) a pre-filter zone comprising at least one introduction channel extending from the inlet to the filter chamber to allow sperm delivered to the fluidic system through the inlet to progress along a fluidic path toward the filter chamber; and
f) a post filter zone comprising a set of separation channels comprising a first separation channel directly connected to the upper portion and a first collection chamber, a second separation channel, and a third separation channel, wherein each separation channel of the set of separation channels, other than the first separation channel is connected to two collection chambers of a set of collection chambers comprising the first collection chamber, a second collection chamber and a third collection chamber, each collection chamber of the set of collection chambers being configured to facilitate harvesting some of motile sperm therein.

In another aspect, provided herein is a system for sorting sperm, comprising:
a) a housing including a lower component and an upper component coupled together;
b) a fluidic system supported by the housing;
c) an inlet providing access to the fluidic system to deliver a sample comprising sperm to the fluidic system;
d) a filter chamber comprising a filter comprising a plurality of pores, and including a lower portion and an upper portion positioned above the lower portion;
e) at least one introduction channel extending from the inlet to the lower portion of the filter chamber to allow sperm delivered to the fluidic system through the inlet to progress along a fluidic path toward the filter chamber; and
f) a post-filter zone comprising:
   i) a first separation channel connecting the upper portion to a first collection chamber, the first collection chamber being configured to facilitate harvesting some motile sperm therein; and
   ii) a second separation channel connected to a second collection chamber, the second collection chamber being configured to facilitate harvesting some motile sperm therein, wherein the second separation channel is connected to either the upper portion or the first collection chamber,
   and in some embodiments, the first separation channel has a length of less than 5 mm, wherein the second separation channel has a length between 5 and 20 mm, and wherein the length of the second separation channel is at least 1.5 times the length of the first separation channel.

In some embodiments, the second separation channel is connected to the first collection chamber.

In another aspect, provided herein is a method for sorting sperm, comprising:
a) delivering a sample of sperm into an inlet connected to a fluidic system;
b) allowing sperm in the sample of sperm to traverse a fluidic path through the fluidic system from the inlet through at least one introduction channel into a filter chamber, wherein in some embodiments, the filter chamber includes a lower portion and an upper portion positioned above the lower portion, and wherein the lower portion and the upper portion are separated by a filter positioned in the filter chamber, wherein the filter has micropores sized to permit a head of the sperm to pass therethrough;
c) without an application of an external force, allowing at least some motile sperm from the sample of sperm that have entered the lower portion of the filter chamber to selectively pass through the filter against gravity, so as to traverse the fluidic path into the upper portion of the filter chamber, and then to traverse the fluidic path through a first separation channel into a first collection chamber such that a percentage of motile sperm present within the first collection chamber is greater than a percentage of motile sperm within the sample; either
d1) allowing at least one of the motile sperm in the first collection chamber to further traverse through a second separation channel into a second collection chamber without the application of an external force; or
d2) allowing at least one of the motile sperm from the upper portion to traverse through a second separation channel connected directly to the upper portion of the filter chamber, into a second collection chamber without the application of an external force, wherein the second separation channel has a length that is at least 1.5× a length of the first separation channel; and
e) harvesting or detecting, and in illustrative embodiments, harvesting at least some of the motile sperm that have passed into one or both the first collection chamber and the second collection chamber.

In another aspect, provided herein is a method for sorting sperm, comprising:
a) delivering a sample comprising sperm to an inlet connected to a fluidic system comprising the inlet, a pre-filter zone comprising at least one introduction channel extending from the inlet to a filter chamber, a filter having a plurality of micropores and arranged in the filter chamber; and a post filter zone comprising a set of separation channels comprising a first separation channel directly connected to an upper portion and a first collection chamber, a second separation channel, and a third separation channel, wherein each separation channel of the set of separation channels, other than the first separation channel is connected to two collection chambers of a set of collection chambers comprising the first collection chamber, a second collection chamber and a third collection chamber, each collection chamber of the set of collection chambers being configured to facilitate harvesting some of motile sperm therein;
b) without an application of external force, allowing sperm delivered to the fluidic system through the inlet to progress along a fluidic path toward the filter chamber, and then to move through the filter to a post-filter zone, and then to move through the post-filter zone to the first collection chamber; and
c) harvesting at least some motile sperm from a collection chamber in the post-filter zone.

In some embodiments, the pre-filter zone is other than a single straight channel. In some embodiments, the filter is arranged horizontally within the filter chamber. In some embodiments, the pre-filter zone comprises a single straight channel. In some embodiments, the pre-filter zone is other than a single straight channel.

In some embodiments, the inlet extends through the upper component, the lower component, or both the upper component and the lower component, the lower chamber extends through the lower component, and/or the upper chamber extends through the upper component.

In some embodiments, the introduction channel and/or the first separation channel is curved or comprises a curved section; and/or the introduction channel and/or the first separation channel comprises one or more 45 to 135 degree bends.

In some embodiments, the sum of lengths of separation channels of the set of separation channels is between 15 mm and 100 mm. In some embodiments, the set of separation channels comprises at least five separation channels and the set of collection chambers comprises at least five collection chambers.

In some embodiments, the set of separation channels comprises a set of parallel channels. In some embodiments, the set of parallel channels comprise a single inlet and/or a single outlet. In some embodiments, the set of separation channels have equal lengths.

In some embodiments, at least one separation channel of the set of separation channels has a different length than another separation channel of the set. In some embodiments, each of the separation channels in the set of separation channels has a length of between about 1 mm and 60 mm, and in some embodiments, wherein each of the separation channels in the set of separation channels has a width of between 0.1 mm and 20 mm. In some embodiments, a combined length of the channels in a sperm swim path in the fluidic system is between 25 mm and 100 mm.

In some embodiments, the second separation channel is a length that is between 1.5× and 25× the length of the first separation channel. In some embodiments, the length of any channel in the set is between 0.5× and 100× the length of any other channel in the set. In some embodiments, the second separation channel is between 2× and 10× longer than the first separation channel. In some embodiments, the width of the first separation channel is between 1 mm and 5 mm. In some embodiments, the width of at least one of the first and second separation channel includes a variable width, and wherein the variable width is one or more of progressively increasing, progressively decreasing, and alternating between increasing and decreasing. In some embodiments, the variable width comprises a width of 0.1 to 5 mm in narrowest width, and between 10 and 20 mm in largest width. In some embodiments, the variable width comprises a width of 0.1 to 2 mm in narrowest width, and between 10 and 20 mm in largest width. In some embodiments, the width of all of the channels in the fluid path is between 1 mm and 10 mm.

In some embodiments, a surface of one of the chambers and/or one of the channels comprises a sperm chemoattractant. In some embodiments, a surface of one or more of the collection chambers in the post filter zone is coated with a sperm chemoattractant. In some embodiments, a surface of one or more of the channels in the post filter zone is coated with a sperm chemoattractant. In some embodiments, the sperm chemoattractant is present on the surface in a concentration gradient. In some embodiments, the sperm chemoattractant is at least one of hyaluronic acid, progesterone, chemokine CCL20, atrial natriuretic peptide (ANP), an odorant, natriuretic peptide type C (NPPC), and allurin.

In some embodiments, the plurality of micropores are sized to permit a head of the sperm to pass therethrough. In some embodiments, the plurality of micropores are between 6 μm and 18 μm in diameter.

In some embodiments, the fluidic system is configured such that motile sperm move through the filter into the upper portion and then move from the upper portion into the first collection chamber without an application of an external force such that a percentage of motile sperm are present within the first collection chamber that is greater than a percentage of motile sperm present within the lower portion. In some embodiments, the fluidic system is configured such that the motile sperm move through the filter into the upper portion, and then move from the upper portion into the first collection chamber, and then move from the first collection chamber into the second collection chamber without the application of an external force such that a percentage of motile sperm present within the second collection chamber is greater than a percentage of motile sperm present in the sample. In some embodiments, the fluidic system is configured such that some of the motile sperm pass through the filter into the upper portion and move from the upper portion into the second collection chamber without the application of an external force.

In some embodiments, at least one of the first collection chamber and the second collection chamber comprises a filter having a plurality of micropores sized to permit a head of the sperm to pass therethrough.

In some embodiments, the housing comprises a support, wherein the support is located under the lower component and forms a lower surface of the at least one introduction channel.

In some embodiments, the filter is a polycarbonate filter.

In some embodiments, motile sperm delivered to the inlet travel a first distance through the first separation channel and a second distance through the second separation channel, wherein the second distance is at least 1.5× greater than the first distance.

In some embodiments, further comprising performing the method a second time with a second fluidic system identical to a first fluidic system, and wherein:
a) when performing the method a first time, motile sperm are collected from the first collection chamber of the first fluidic system; and
b) when performing the method the second time, motile sperm are collected from the second collection chamber of the second fluidic system.

In some embodiments, at least 1000× more motile sperm are collected from the first collection chamber of the first fluidic system, than are collected from the second collection chamber of the second fluidic system.

In some embodiments, fluidic system comprises a housing including a lower component and an upper component coupled together; wherein the inlet extends through the lower component and provides access to the fluidic system to deliver sperm to the fluidic system.

In some embodiments, the filter is located between the upper component and the lower component.

In some embodiments, a top of the upper portion of the filter chamber is an open outlet that provides access to the fluidic system as the motile sperm traverse the fluidic path through the fluidic system from the inlet to the filter chamber. In some embodiments, the at least some of the motile sperm are harvested, a higher percentage of motile sperm are present within the first collection chamber than in the sample.

In some embodiments, atop portion of the upper portion of the filter chamber, atop portion of the first collection chamber and a top portion of the second collection chamber are open outlets that provide access to the fluidic system.

In some embodiments, a surface of one or both of the first collection chamber and the second collection chamber comprises a sperm chemoattractant so as to provide one or more sperm chemoattractant-coated surfaces, and wherein the method further comprises before the harvesting, allowing at least one of the motile sperm in one or both the first collection chamber and the second collection chamber to bind the one or more sperm chemoattractant-coated surfaces.

In some embodiments, the harvesting is performed by collecting at least one of the motile sperm bound to the sperm chemoattractant-coated surfaces.

In some embodiments, at least 1000× more motile sperm are collected from the first collection chamber than the second collection chamber.

In some embodiments, the first collection chamber is connected to the second collection chamber by the second separation channel.

In some embodiments, the at least one of the motile sperm from the upper portion traverse through the second separation channel connected directly to the upper portion, into the second collection chamber without an application of an external force, wherein the second separation channel is between 1.5× to 10× a length of a first fluidic connection channel.

In some embodiments, the method is performed at least two times, a first time using a first fluidic system and a second time using a second fluidic system, wherein the first fluidic system and the second fluidic system are identical, and wherein one of the first time or the second time at least 1000 sperm are collected from the first collection chamber and the other of the first time or the second time 1 to 100 sperm are collected from the second collection chamber. In some embodiments, the method is performed at least two times, a first time using a first fluidic system and a second time using a second fluidic system, wherein the first fluidic system and the second fluidic system are identical, and wherein one of the first time or the second time at least 1000 sperm are collected from the first collection chamber and the other of the first time or the second time 1 to 100 sperm are collected from the most distal collection chamber.

In some embodiments, 1,000,000 or more sperm are harvested from one collection chamber and 100,000 or fewer sperm are harvested from a different collection chamber during the same performance of the method.

In some embodiments, a dam is present in a collection chamber or a separation channel to block sperm from traveling past the dam. In some embodiments, sperm are concentrated at the collection chamber before the dam. In some embodiments, a dam is present at a junction of the first collection chamber and the second separation channel, or in the second separation channel to concentrate sperm in the first collection chamber.

In some embodiments, the method further comprises performing an assisted reproductive technology (ART) procedure using at least one of the sperm that are harvested. In some embodiments, the ART procedure is chosen based on the number of sperm harvested from one or more collection chambers. In some embodiments, the ART procedure is selected from an intrauterine insemination (IUI), an in vitro fertilization (IVF), and an intracytoplasmic sperm injection (ICSI).

In some embodiments, at least 1,000,000 sperm are harvested and the ART procedure is IUI. In some embodiments, less than 1,000,000 sperm are harvested and the ART procedure is in vitro fertilization (conventional insemination). In some embodiments, less than 10,000 sperm are harvested and the ART procedure is ICSI.

In some embodiments, the post filter zone comprises between 3 and 8 collection chambers. In some embodiments, between 3 and 7 of the collection chambers are each connected through their corresponding separation channel, to a first collection chamber that is connected to the filter chamber by a first separation channel.

In some embodiments, the lower component is a lower layer, the upper component is an upper layer, or both the lower component and the upper component are a lower layer and upper layer, respectively. In some embodiments, the lower component is a lower component layer, and wherein the upper component is an upper component layer. In some embodiments, the lower component layer and/or the upper component layer are between 2 and 25 mm, 2 and 20 mm, 2 and 15 mm, 3 and 12 mm, 3 and 10 mm, 3 and 8 mm, or 4 and 10 mm thick. In some embodiments, the housing, the lower component, the upper component, and/or the support have a rectangular shape, a width of between 1.5 cm and 5 cm, and a length of between 3 cm and 15 cm.

In some embodiments, the inlet extends through the lower component, the upper component, or both the lower component and the upper component. In some embodiments, the lower portion of the filter chamber extends through lower component, the upper portion for the filter chamber extends through the upper component, or both the lower portion of the filter chamber extends through the lower component and the upper portion of the filter chamber extends through the upper component.

In some embodiments, the filter chamber comprises at least two filters. In some embodiments, the at least two filters comprise a plurality of micropores of between 6 μm and 18 μm in diameter. In some embodiments, the at least two filters comprise a first filter with a plurality of micropores having a first diameter, and a second filter with a plurality of micropores having a second diameter different than the first diameter.

In some embodiments, at least one of the first collection chamber and the second collection chamber are adapted for insertion of a pipette tip therein.

In some embodiments, the sperm are present in the inlet and wherein the top end of the filter chamber is open to the atmosphere. In some embodiments, sperm are within the at least one introduction channel but not above the filter. In some embodiments, at least one introduction channel, the first collection chamber and the second collection chamber do not comprise a filter.

In some embodiments, no other channels that are open outlets or are connected to one or more open outlets are directly connected to the upper portion other than the separation channel or channels.

In some embodiments, the first collection chamber and the second collection chamber do not comprise a filter.

In some embodiments, at least 100,000 sperm are harvested from at least 2 collection chambers during the same performance of the method. In some embodiments, 100,000 or more sperm are harvested from each of at least 2 collection chambers. In some embodiments, 100,000 or more sperm are harvested from each of at least 3 collection chambers. In some embodiments, at least 100,000 sperm are harvested from a collection chamber that is proximal to the filter chamber compared to a collection chamber from which less than 100,000 sperm are harvested. In some embodiments, at least 1,000,000 sperm are harvested from a collection chamber that is proximal to the filter chamber compared to a collection chamber from which less than 100,000 sperm are harvested.

In some embodiments, the sperm chemoattractant is within a hydrogel that is associated with the surface of the one or the chambers and/or the one of the channels. In some embodiments, the sperm chemoattractant is released as the sperm traverse or progress along the fluidic path through the fluidic system.

In some embodiments, the first separation channel has a length of between 1 to 5 mm. In some embodiments, the second separation channel has a length of between about 10 mm and 60 mm.

In some embodiments, the pre-filter zone is configured and/or adapted to sort sperm based on their swimming ability and is other than a single straight channel.

In some embodiments, the post-filter zone is configured and/or adapted to sort sperm based on their swimming ability and is other than a single straight separation channel connected to a single collection chamber, and/or wherein the filter is arranged in the filter chamber to cause sperm traveling along the fluidic path to move through the filter and against gravity to reach the upper portion.

In some embodiments, the pre-filter zone is configured and/or adapted to sort sperm based on their swimming ability and is other than a single straight channel and wherein the post filter zone is configured and/or adapted to sort sperm based on their swimming ability and is other than a single straight separation channel connected to a single collection chamber.

In some embodiments, each of the separation channels in the set of separation channels has a length of between about 1 mm and 60 mm, and wherein the width of each of the separation channels is between 0.1 mm and 20 mm.

In some embodiments, the second separation channel is between 1.5× and 25× the length of the first separation channel.

In some embodiments, the method is performed at least two times, a first time using the first fluidic system and a second time using a second fluidic system, wherein the first fluidic system and the second fluidic system are identical, and wherein one of the times at least 1000 sperm are collected from the first collection chamber and the other time 1 to 100 sperm are collected from the second collection chamber. In some embodiments, the method is performed at least two times, a first time using the first fluidic system and a second time using a second fluidic system, wherein the first fluidic system and the second fluidic system are identical, and wherein one of the times at least 1000 sperm are collected from the first collection chamber and the other time 1 to 100 sperm are collected from the most distal collection chamber.

In some embodiments, the method further comprises performing an assisted reproductive technology (ART) procedure using at least one of the harvested sperm. In some embodiments, the ART procedure is chosen based on the number of sperm harvested from one or more collection chambers.

Illustrative embodiments have been discussed, and it should be appreciated that many equivalents, alternatives, variations, and modifications, aside from those expressly stated, are possible and within the scope of the invention.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as discussed may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments discussed may be made by skilled artisans without changing the fundamental aspects of the present disclosure. Any of the disclosed embodiments can be used in combination with any other disclosed embodiment.

In some instances, some concepts have been discussed with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

1-76. (canceled)

77. A system for sorting sperm, the system comprising:
a) a housing including a lower component and an upper component coupled together;
b) a fluidic system supported by the housing;
c) an inlet providing access to the fluidic system to deliver a sample comprising sperm to the fluidic system;
d) a filter chamber comprising a filter comprising a plurality of pores, a lower portion, and an upper portion positioned above the lower portion;
e) at least one introduction channel extending from the inlet to the lower portion of the filter chamber to allow sperm delivered to the fluidic system through the inlet to progress along a fluidic path toward the filter chamber; and
f) a post-filter zone comprising a set of separation channels and a set of collection chambers, the post-filter zone comprising:
i) a first separation channel of the set of separation channels connecting the upper portion to a first collection chamber of the set of collection chambers, the first collection chamber being configured to facilitate harvesting some motile sperm therein; and
ii) a second separation channel of the set of separation channels connected to a second collection chamber of the set of collection chambers, the second collection chamber being configured to facilitate harvesting some motile sperm therein, wherein the second separation channel is connected to the upper portion or the first collection chamber.

78. The system of claim 77, wherein the first separation channel has a length of less than 5 mm, wherein the second separation channel has a length between 5 and 20 mm, and wherein the length of the second separation channel is at least 1.5 times the length of the first separation channel.

79. The system of claim 77, wherein the post-filter zone comprises a third separation channel of the set of separation channels, and wherein each of the second separation channel and the third separation channel is connected to two of the first collection chamber, the second collection chamber, and a third collection chamber of the set of collection chambers.

80. The system of claim 77, wherein the second separation channel is connected to the first collection chamber.

81. The system of claim 77, wherein the filter is arranged horizontally within the filter chamber, and wherein at least some motile sperm from the sample are configured pass through the filter against gravity.

82. The system of claim 77, wherein the introduction channel is a straight channel.

83. The system of claim 77, wherein the introduction channel and/or the first separation channel is curved or comprises a curved section; and/or wherein the introduction channel and/or the first separation channel comprises one or more 45 to 135 degree bends.

84. The system of claim 77, wherein the set of separation channels comprises a set of parallel channels.

85. The system of claim 84, wherein the set of parallel channels comprise a single inlet and/or a single outlet.

86. The system of claim 77, wherein a surface of one collection chamber of the set of collection chambers and/or one separation channel of the set of separation channels comprises a sperm chemoattractant.

87. The system of claim 86, wherein the sperm chemoattractant is present on the surface in a concentration gradient.

88. The system of claim 77, wherein the housing comprises a support disposed under the lower component and forming a lower surface of the at least one introduction channel.

89. The system of claim 77, comprising a dam configured to block sperm from traveling past the dam, and wherein sperm are concentrated at one or more collection chamber of the set of collection chambers before the dam.

90. The system of claim 77, wherein the lower portion of the filter chamber extends through lower component, the upper portion for the filter chamber extends through the upper component, or both the lower portion of the filter chamber extends through the lower component and the upper portion of the filter chamber extends through the upper component.

91. A method for sorting sperm, the method comprising:
a) delivering a sample of sperm into an inlet of a fluidic system;
b) allowing sperm in the sample of sperm to traverse a fluidic path through the fluidic system from the inlet through at least one introduction channel into a filter chamber, wherein the filter chamber includes a lower portion, an upper portion positioned above the lower portion, and a filter between the lower portion and the upper portion, wherein the filter comprises pores sized to permit a head of the sperm to pass therethrough;
c) without an application of an external force, allowing at least some motile sperm from the sample of sperm that have entered the lower portion of the filter chamber to selectively pass through the filter against gravity, so as to traverse the fluidic path into the upper portion of the filter chamber, and then to traverse the fluidic path through a first separation channel into a first collection chamber such that a percentage of motile sperm present within the first collection chamber is greater than a percentage of motile sperm within the sample of sperm;
either
d1) allowing at least one of the motile sperm in the first collection chamber to further traverse through a second separation channel into a second collection chamber without the application of an external force; or
d2) allowing at least one of the motile sperm from the upper portion to traverse through a second separation channel connected directly to the upper portion of the filter chamber, into a second collection chamber without the application of an external force, wherein the second separation channel has a length that is at least 1.5 times a length of the first separation channel;
and
e) harvesting at least some of the motile sperm that have passed into one or both the first collection chamber and the second collection chamber, wherein a higher percentage of motile sperm are present within the one or both the first collection chamber and the second collection chamber than in the sample of sperm.

92. The method of claim 91, wherein a surface of one or both of the first collection chamber and the second collection chamber comprises a sperm chemoattractant so as to provide one or more sperm chemoattractant-coated surfaces, the method comprising:
allowing the at least one of the motile sperm in one or both the first collection chamber and the second collection chamber to bind the one or more sperm chemoattractant-coated surfaces, and wherein harvesting comprises collecting at least one of the motile sperm bound to the sperm chemoattractant-coated surfaces.

93. The method of claim 91, wherein harvesting comprises collecting the at least some of the motile sperm from one or more of an open outlet of the upper portion of the filter chamber, an open outlet of the first collection chamber, and an open outlet of the second collection chamber.

94. A system for sorting sperm, the system comprising:
a) a filter chamber comprising a lower portion, an upper portion positioned above the lower portion, and a filter arranged between the lower portion and the upper portion, the filter comprising a plurality of pores; and
b) a post filter zone comprising a set of separation channels comprising a first separation channel directly connected to an upper portion and a first collection chamber, a second separation channel, and a third separation channel, wherein each separation channel of the set of separation channels, other than the first separation channel is connected to two collection chambers of a set of collection chambers comprising the first collection chamber, a second collection chamber and a third collection chamber, each collection chamber of the set of collection chambers being configured to facilitate harvesting some of motile sperm therein.

95. The system of claim 94, wherein the system further comprises an inlet connected either directly or indirectly to the lower portion of the filter chamber.

96. The system of claim 94, wherein a width of at least one of the first and second separation channel includes a variable width, and wherein the variable width is one or more of progressively increasing, progressively decreasing, and alternating between increasing and decreasing.
